(19) United States
(12) Patent Application Publication
(10) Pub. No.: US 2021/0155771 A1
(43) Pub. Date: May 27, 2021

(54) PLASTICIZER AND PLASTIC PRODUCT

(71) Applicant: LARGAN MEDICAL CO., LTD., Taichung City (TW)

(72) Inventors: Wei-Yuan CHEN, Taichung City (TW); Tzu-Rong LU, Taichung City (TW); Yi-Ling CHEN, Taichung City (TW); Chun-Hung TENG, Taichung City (TW)

(21) Appl. No.: 17/102,617

(22) Filed: Nov. 24, 2020

Related U.S. Application Data
(60) Provisional application No. 62/939,733, filed on Nov. 25, 2019.

(51) Int. Cl. C08K 5/00; C08K 5/17; C08L 33/06

(52) U.S. Cl. CPC C08K 5/0016; C08L 33/06; C08K 5/175

(57) ABSTRACT

A plasticizer, which is biodegradable, has a molecule including a central structure, at least two connecting structures and at least one side-chain structure. The central structure includes at least one of a benzene derivative and at least one amino acid. The connecting structures are respectively connected to the central structure, wherein the connecting structures include a first connecting structure and a second connecting structure. The first connecting structure is an amine group, and the second connecting structure is a carboxyl group. The side-chain structure is a chain of multiple carbon atoms, and the side-chain structure is connected to at least one of the first connecting structure and the second connecting structure. An amide bond is formed as the side-chain structure connected to the amine group, and an ester bond is formed as the side-chain structure connected to the carboxyl group.

### RELATED APPLICATIONS

This application claims priority to U.S. Provisional Application Ser. No. 62/939,733, filed Nov. 25, 2019, which is herein incorporated by reference.

### BACKGROUND

### Technical Field

The present disclosure relates to an additive for plastic materials. More particularly, the present disclosure relates to a biodegradable plasticizer.

### Description of Related Art

In order to solve the environmental problems brought on by plastic products, biodegradable plastics, such as polylactic acid (PLA), poly(butylene succinate) (PBS) and poly(butylene adipate-co-terephthalate) (PBAT), have already been developed. However, properties of this type of plastics are still not as good as petroleum-based plastics. For example, polylactic acid has excellent rigidity and high transparency, but lacks enough durability, heat resistance, impact resistance, flexibility and airtightness, which limits the applications of polylactic acid.

To deal with problems in properties, textures and costs of biodegradable plastics, conventional biodegradable additives can be added to change the flexibility and durability of biodegradable plastics. However, the enhancement of biodegradation in properties of biodegradable plastics made by adding conventional additives is still insufficient. Also, the conventional additives are likely to release and have higher cost, so there has a lot of improvements for the conventional additives. Furthermore, the conditions for degradation of biodegradable plastics are tough. For example, plastic products made of polylactic acid can completely degrade into carbon dioxide and water only under particular environmental conditions for a sufficient time, and then enter the cycle without harming the natural environment. However, it is pretty difficult for plastic products of polylactic acid naturally and completely degrading in a typical environment. In this regard, the important trend for future is to develop an additive, which can enhance the degradation rate to facilitate biodegradable plastics quickly degrading and entering the natural environment.

### SUMMARY

According to one aspect of the present disclosure, a plasticizer, which is biodegradable, has a molecule including a central structure, at least two connecting structures and at least one side-chain structure. The central structure includes at least one of a benzene derivative and at least one amino acid. The at least two connecting structures are respectively connected to the central structure, wherein the at least two connecting structures include a first connecting structure and a second connecting structure. The first connecting structure is an amine group, and the second connecting structure is a carboxyl group. The at least one side-chain structure is a chain of multiple carbon atoms, and the at least one side-chain structure is connected to at least one of the first connecting structure and the second connecting structure. An amide bond is formed as the at least one side-chain structure connected to the amine group, and an ester bond is formed as the at least one side-chain structure connected to the carboxyl group. When a number of carbon atoms of the side-chain structure which is connected to the amine group is nNC, and a number of carbon atoms of the side-chain structure which is connected to the carboxyl group is nOC, the following condition is satisfied: 0≤nNC/(nNC+nOC)≤1.00, wherein each of nNC and nOC is 0 or positive integer, but nNC and nOC are not 0 at the same time.

According to another aspect of the present disclosure, a plastic product, which is biodegradable, includes the plasticizer of the aforementioned aspect and a polyester plastic, wherein the polyester plastic is a polylactic acid, a polybutylene succinate, a poly(butylene adipate-co-terephthalate) or a polyhydroxyalkanoate, and the plasticizer is mixed with the polyester plastic.

According to another aspect of the present disclosure, a plasticizer, which is biodegradable, has a molecule including at least two central structures, at least two connecting structures and a side-chain structure. Each of the central structures includes at least one of a benzene derivative and at least one amino acid. Each of the connecting structures is a carboxyl group, and each of the central structures is connected to at least one of the connecting structures. The side-chain structure is a chain of multiple carbon atoms, each of the central structures is connected to the side-chain structure through the connecting structures, and an ester bond is formed as the side-chain structure connected to each of the carboxyl groups.

### BRIEF DESCRIPTION OF THE DRAWINGS

The present disclosure can be more fully understood by reading the following detailed description of the embodiment, with reference made to the accompanying drawings as follows:

FIG. 1 is a structural schematic view of a molecule of a plasticizer according to the let embodiment of the present disclosure.

FIG. 2 is a structural schematic view of a molecule of a plasticizer according to the 2nd embodiment of the present disclosure.

FIG. 3 is a structural schematic view of a molecule of a plasticizer according to the 3rd embodiment of the present disclosure.

FIG. 4 is a structural schematic view of a molecule of a plasticizer according to the 4th embodiment of the present disclosure.

FIG. 5 is a structural schematic view of a molecule of a plasticizer according to the 5th embodiment of the present disclosure.

FIG. 6 is a structural schematic view of a molecule of a plasticizer according to the 6th embodiment of the present disclosure.

FIG. 7 is a structural schematic view of a molecule of a plasticizer according to the 7th embodiment of the present disclosure.

FIG. 8 is a structural schematic view of a molecule of a plasticizer according to the 8th embodiment of the present disclosure.

FIG. 9 is a structural schematic view of a molecule of a plasticizer according to the 9th embodiment of the present disclosure.

FIG. 10 is a structural schematic view of a molecule of a plasticizer according to the 10th embodiment of the present disclosure.

FIG. 11 is a structural schematic view of a molecule of a plasticizer according to the 11th embodiment of the present disclosure.

FIG. 12 is a structural schematic view of a molecule of a plasticizer according to the 12th embodiment of the present disclosure.

FIG. 13 is a structural schematic view of a molecule of a plasticizer according to the 13th embodiment of the present disclosure.

FIG. 14 is a structural schematic view of a molecule of a plasticizer according to the 14th embodiment of the present disclosure.

FIG. 15 shows results of biodegradation tests of the comparison, the 29th Example and the 30th Example.

### DETAILED DESCRIPTION

A plasticizer, which is biodegradable, has a molecule including a central structure, at least two connecting structures and at least one side-chain structure. The at least two connecting structures are respectively connected to the central structure, and the side-chain structure is connected to at least one of the at least two connecting structures.

The central structure includes at least one of a benzene derivative and at least one amino acid. It is favorable for maintaining the biodegradability of the plasticizer by having the benzene derivative. The amino acid can be degraded and metabolized in animal cells, so that it is favorable for enhancing the biodegradability of the plasticizer. Moreover, the central structure can be the benzene derivative, and can further include the amino acid. Furthermore, the central structure can be the amino acid, and can include at least two of the amino acid. The residue of the amino acid can be acidic, basic, hydrophilic or hydrophobic, so that it is favorable for adjusting the properties of the plasticizer according to the residue characteristics of the amino acid.

The benzene derivative can be compounds with similar structures, such as 2-aminobenzoic acid, 3-aminobenzoic acid, 4-aminobenzoic acid, 2-hydroxybenzoic acid, 3-hydroxybenzoic acid or 4-hydroxybenzoic acid.

The amino acid can be standard amino acids, essential amino acids which humans cannot synthesize, modified amino acids (such as hydroxyproline, hydroxylysine or thyroxine) or other non-standard amino acids (such as homocysteine, homoserine or ornithine). The standard amino acids and essential amino acids are listed in Table 1 below.

TABLE 1
Standard Amino Acids and Essential Amino Acids

| Amino Acid | Abbreviation | Essential Amino Acid |
|---|---|---|
| Alanine | Ala | No |
| Arginine | Arg | No |
| Asparagine | Asn | No |
| Aspartate | Asp | No |
| Cysteine | Cys | No |
| Glutamate | Glu | No |
| Glutamine | Gln | No |
| Glycine | Gly | No |
| Histidine | His | Yes |
| Isoleucine | Ile | Yes |
| Leucine | Leu | Yes |
| Lysine | Lys | Yes |
| Methionine | Met | Yes |
| Phenylalanine | Phe | Yes |
| Proline | Pro | No |
| Pyrrolysine | Pyl | No |
| Selenocysteine | Sec | No |
| Serine | Ser | No |
| Threonine | Thr | Yes |
| Tryptophan | Trp | Yes |
| Tyrosine | Tyr | No |
| Valine | Val | Yes |

The central structure can also be glycerol (propane-1,2,3-triol) or 2-(2-hydroxyethoxy)propan-1-ol.

The at least two connecting structures include a first connecting structure and a second connecting structure. The first connecting structure is an amine group, and the second connecting structure is a carboxyl group.

If the central structure is the benzene derivative, the position of the amine group and the position of the carboxyl group can be adjacent to each other (ortho) or not adjacent (meta or para) on the benzene derivative.

The amine group can be a functional group of —NH₂, —NHR, or —NR₂. Moreover, the amine group can be a primary amine, a secondary amine, a tertiary amine or a quaternary ammonium cation. Furthermore, a hydroxyl group can be substituted for the amine group of the first connecting structure. When the side-chain structure is connected to the hydroxyl group, the connection therebetween can be split through hydrolysis reaction, so that it is favorable for enhancing the biodegradability of the plasticizer.

When the molecule of the plasticizer includes two connecting structures, the two connecting structures are respectively the first connecting structure and the second connecting structure. Moreover, the molecule of the plasticizer can include three connecting structures, which are respectively the first connecting structure, the second connecting structure and a third connecting structure. The third connecting structure can be the residue of the amino acid. For example, the amine group of the residue from lysine can be taken as the third connecting structure, or the carboxyl group of the residue from aspartate can be taken as the third connecting structure. Further, the molecule of the plasticizer can include four connecting structures, which are respectively the first connecting structure, the second connecting structure, the third connecting structure and a fourth connecting structure. The third connecting structure and the fourth connecting structure can be the same functional groups, such as the binary amine groups of the residues from arginine, or can be different functional groups. Furthermore, when the central structure includes a plurality of amino acids, the molecule of the plasticizer can include more than five connecting structures.

The side-chain structure is a chain of multiple carbon atoms. The number of carbon atoms of the side-chain structure can be 1, 2, 3, 4, 5, 6, 7, 8, 9, 10, 11, 12, 13, 14, 15, 16, 17, 18, 19 or 20, etc. Moreover, the side-chain structure can include an oxygen atom to form an ether group, and the number of oxygen atoms of the side-chain structure can be 1, 2, 3, 4, 5, 6 or 7, etc.

The side-chain structure can be a saturated fatty acid. The number of carbon atoms of the saturated fatty acid can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36, such as acetic acid (lipid number C2:0), butyric acid (C4:0), hexanoic acid (C6:0), octanoic acid (C8:0), decanoic acid (C10:0), lauric acid (C12:0), lignoceric acid (C24:0), or hexatriacontanoic acid (C36:0).

The side-chain structure can be an unsaturated fatty acid. The number of carbon atoms of the unsaturated fatty acid can be 14, 15, 16, 17, 18, 19, 20, 21, 22, 23 or 24, and the number of double bonds of the unsaturated fatty acid can be 1, 2, 3, 4, 5 or 6, such as paullinic acid (020:1), dihomo-γ-linolenic acid (DGLA, C20:3), docosahexaenoic acid (DHA, C22:6), or tetracosenoic acid (C24:1).

The side-chain structure can be formed by continuous polymerization with ethylene glycol, polyethylene glycol (PEG), propylene glycol or polypropylene glycol (PPG) as monomers. Moreover, the side-chain structure can be formed by polymerization of the monomers selected from the group consisting of ethylene glycol, polyethylene glycol, propylene glycol and polypropylene glycol. The side-chain structure is formed by polymerization of the abovementioned monomers, the required molecular weight of the plasticizer can be controlled, and the hydrophilicity and hydrophobicity of the plasticizer are properly adjusted, so that it is favorable for the plasticizer performing the superior characteristics and enhancing the compatibility of the plasticizer mixing with the plastic.

The side-chain structure is connected to at least one of the first connecting structure and the second connecting structure, that is, the central structure is connected to the side-chain structure through the first connecting structure and/or the second connecting structure by a method of esterification, dehydration or peptide synthesis.

When the side-chain structure is connected to the amine group, an amide bond is formed, so that it is favorable for enhancing the efficiency of the plasticizer degraded by the enzyme, and further improving the biodegradability of the plasticizer. Moreover, when the side-chain structure is connected to the carboxyl group, an ester bond is formed. Furthermore, when the side-chain structure is connected to the hydroxyl group, an ether bond is formed. The ether bond can be split through hydrolysis reaction in a particular environment, so that it is favorable for enhancing the biodegradability of the plasticizer.

The plasticizer can further include at least one polar functional group connected to the side-chain structure, and the polar functional group can be located in the middle or at the end of the side-chain structure. Moreover, the polar functional group can be a carboxyl group (—COOH), a hydroxyl group (—OH), an amine group (—NH₂) or a phosphoryl group (—PO(OH)₂). Furthermore, the plasticizer can include at least two of the polar functional group, which are selected from the group consisting of the carboxyl group, the hydroxyl group, the amine group and the phosphoryl group. The polar functional group is arranged according to different requirements, so that it is favorable for enhancing the hydrophilicity of the plasticizer and the compatibility between the plasticizer and the plastic, which helps the plasticizer mix with the plastic.

When a number of carbon atoms of the side-chain structure which is connected to the amine group is nNC, and a number of carbon atoms of the side-chain structure which is connected to the carboxyl group is nOC, the following condition is satisfied: 0≤nNC/(nNC+nOC)≤1.00, wherein each of nNC and nOC is 0 or positive integer, but nNC and nOC are not 0 at the same time. Moreover, the following conditions can be satisfied: 0.01≤nNC/(nNC+nOC); nNC/(nNC+nOC)≤1.00; 0.05≤nNC/(nNC+nOC)≤0.95; 0.10≤nNC/(nNC+nOC)≤0.90; 0.15≤nNC/(nNC+nOC)≤0.80; 0.20≤nNC/(nNC+nOC)≤0.70; 0.25≤nNC/(nNC+nOC)≤0.60; 0.30≤nNC/(nNC+nOC)≤0.50; or 0.35≤nNC/(nNC+nOC)≤0.40.

A number of oxygen atoms of the side-chain structure which is connected to the amine group is nNO. A number of oxygen atoms of the side-chain structure which is connected to the carboxyl group is nOO. A number of carbon atoms of the side-chain structure which is connected to the hydroxyl group is nO′C. A number of oxygen atoms of the side-chain structure which is connected to the hydroxyl group is nO′O. nNC and nNO of the side-chain structure are counted from the nitrogen atom of the amine group. nOC, nOO, nO′C and nO′O of the side-chain structure are counted from the oxygen atom of the carboxyl group or the hydroxyl group. The carbon atoms or the oxygen atoms in the polar functional group are not included as calculating nNC, nNO, nOC, nOO, nO′C and nO′O. nO′C is taken as nOC and nO′O is taken as nOO during calculation.

When the molecular weight of the plasticizer is MwP, the following condition is satisfied: 450 Dalton ≤MwP. As the molecular weight of the plasticizer is larger, it is more difficult for the plasticizer to pass through the cell membrane, so that it is favorable for preventing the plasticizer directly passing through the cell membrane into the cell. Moreover, the following conditions can be satisfied: MwP≤2000 Dalton; 475 Dalton ≤MwP≤1000 Dalton; 500 Dalton ≤MwP; 525 Dalton ≤MwP; 550 Dalton ≤MwP; or 600 Daltons ≤MwP≤1500 Dalton.

Each of the aforementioned features of the plasticizer can be utilized in various combinations for achieving the corresponding effects.

According to another aspect of the present disclosure, a plasticizer, which is biodegradable, has a molecule including at least two central structures, at least two connecting structures and a side-chain structure. Each of the central structures is connected to at least one of the connecting structures. Each of the central structures is connected to the side-chain structure through the connecting structures, that is, the central structures are all connected to the same side-chain structure. The detailed features of the central structures, the connecting structures and the side-chain structure are the same as the abovementioned plasticizer, and an explanation in this regard will not be provided again.

According to the present disclosure, a plastic product is provided, wherein the plastic product is biodegradable. The plastic product includes the aforementioned plasticizer and a polyester plastic, wherein the polyester plastic can be a polylactic acid, a polybutylene succinate, a poly(butylene adipate-co-terephthalate) or a polyhydroxyalkanoate (PHA), and the plasticizer is mixed with the polyester plastic. The type of the polyester plastic is chosen to ensure that the plasticizer is mixed with the plastic of high compatibility, so that it is favorable for enhancing the ductility of the plastic and reducing the possibility of releasing of the plasticizer. Moreover, the polyhydroxyalkanoate can be a polyhydroxybutyrate (PHB).

### 1st Embodiment

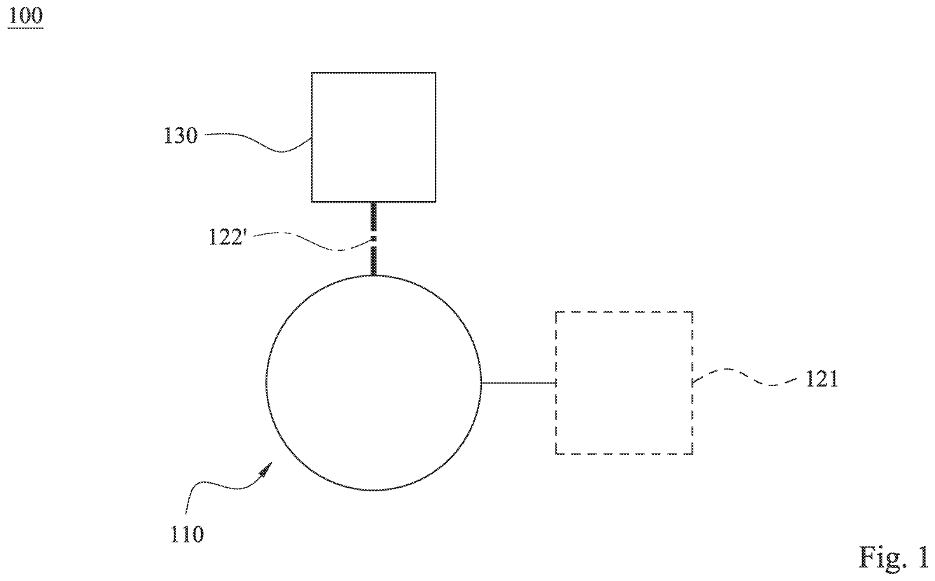

FIG. 1 is a structural schematic view of a molecule of a plasticizer 100 according to the 1st embodiment of the present disclosure. In FIG. 1, the molecule of the plasticizer 100 according to the 1st embodiment includes a central structure 110, two connecting structures and a side-chain structure 130.

The central structure 110 is a benzene derivative.

The two connecting structures are a first connecting structure and a second connecting structure and connected to the central structure 110, respectively. The first connecting structure is an amine group 121, and the second connecting structure is a carboxyl group (not shown in drawings).

The side-chain structure 130 is formed by polymerization with ethylene glycol or polyethylene glycol as monomers. The side-chain structure 130 is connected to the second connecting structure to form an ester bond 122′.

### 2nd Embodiment

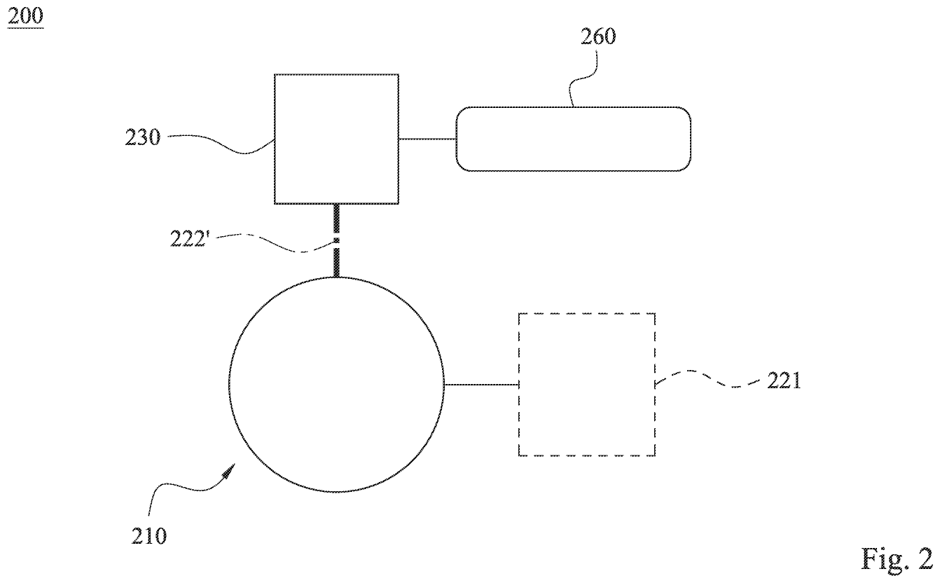

FIG. 2 is a structural schematic view of a molecule of a plasticizer 200 according to the 2nd embodiment of the present disclosure. In FIG. 2, the molecule of the plasticizer 200 according to the 2nd embodiment includes a central structure 210, two connecting structures, a side-chain structure 230 and a polar functional group 260.

The central structure 210 is a benzene derivative.

The two connecting structures are a first connecting structure and a second connecting structure and connected to the central structure 210, respectively. The first connecting structure is an amine group 221, and the second connecting structure is a carboxyl group (not shown in drawings).

The side-chain structure 230 is formed by polymerization with propylene glycol or polypropylene glycol as monomers. The side-chain structure 230 is connected to the second connecting structure to form an ester bond 222′.

The polar functional group 260 is connected to the side-chain structure 230.

### 3rd Embodiment

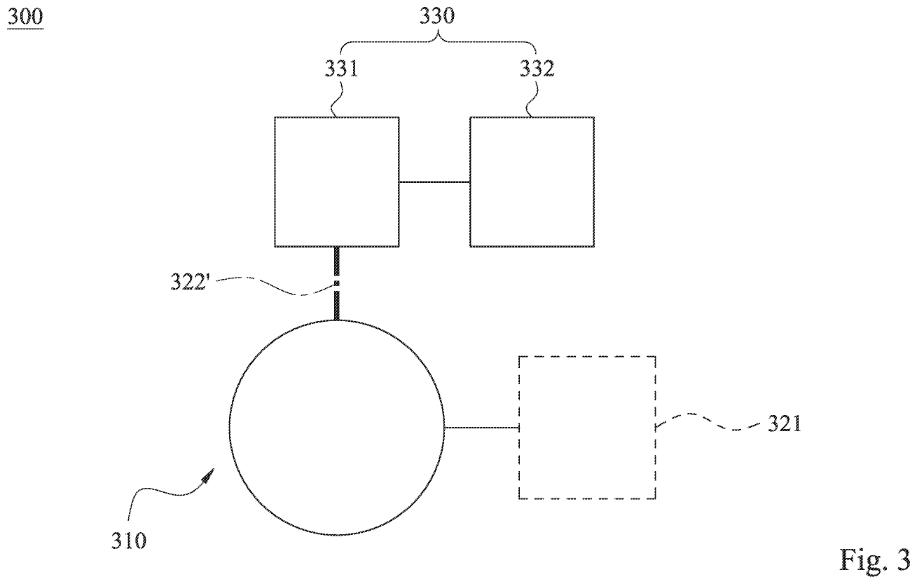

FIG. 3 is a structural schematic view of a molecule of a plasticizer 300 according to the 3rd embodiment of the present disclosure. In FIG. 3, the molecule of the plasticizer 300 according to the 3rd embodiment includes a central structure 310, two connecting structures and a side-chain structure 330.

The central structure 310 is a benzene derivative.

The two connecting structures are a first connecting structure and a second connecting structure and connected to the central structure 310, respectively. The first connecting structure is an amine group 321, and the second connecting structure is a carboxyl group (not shown in drawings).

The side-chain structure 330 is formed by a first short chain 331 connected to a second short chain 332. The first short chain 331 is formed by polymerization with ethylene glycol or polyethylene glycol as monomers. The second short chain 332 is formed by polymerization with propylene glycol or polypropylene glycol as monomers. The side-chain structure 330 is connected to the second connecting structure to form an ester bond 322′.

### 4th Embodiment

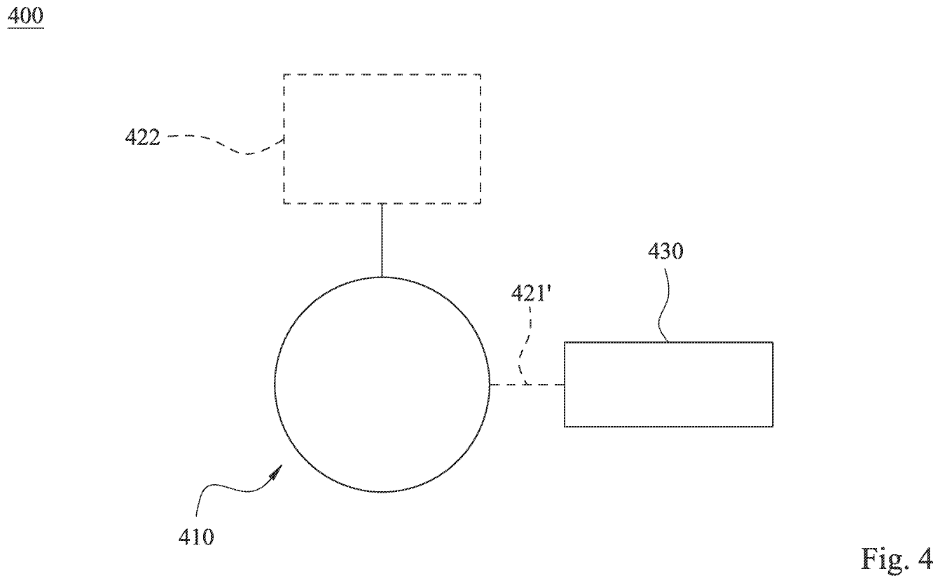

FIG. 4 is a structural schematic view of a molecule of a plasticizer 400 according to the 4th embodiment of the present disclosure. In FIG. 4, the molecule of the plasticizer 400 according to the 4th embodiment includes a central structure 410, two connecting structures and a side-chain structure 430.

The central structure 410 is a benzene derivative.

The two connecting structures are a first connecting structure and a second connecting structure and connected to the central structure 410, respectively. The first connecting structure is an amine group (not shown in drawings), and the second connecting structure is a carboxyl group 422.

The side-chain structure 430 is a fatty acid. The side-chain structure 430 is connected to the first connecting structure to form an amide bond 421′.

### 5th Embodiment

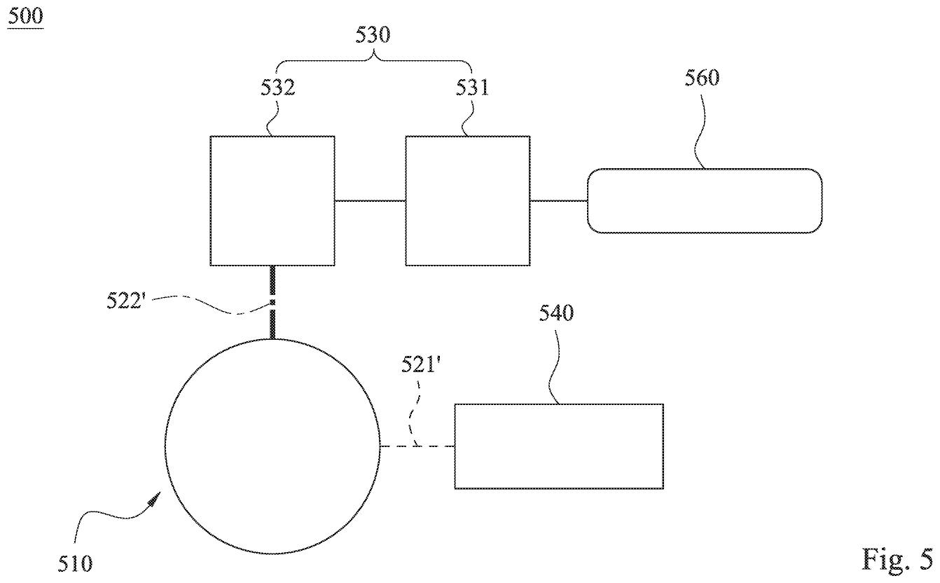

FIG. 5 is a structural schematic view of a molecule of a plasticizer 500 according to the 5th embodiment of the present disclosure. In FIG. 5, the molecule of the plasticizer 500 according to the 5th embodiment includes a central structure 510, two connecting structures, two side-chain structures 530, 540 and a polar functional group 560.

The central structure 510 is a benzene derivative.

The two connecting structures are a first connecting structure and a second connecting structure and connected to the central structure 510, respectively. The first connecting structure is an amine group (not shown in drawings), and the second connecting structure is a carboxyl group (not shown in drawings).

The side-chain structure 530 is formed by a first short chain 531 connected to a second short chain 532. The first short chain 531 is formed by polymerization with ethylene glycol or polyethylene glycol as monomers. The second short chain 532 is formed by polymerization with propylene glycol or polypropylene glycol as monomers. The side-chain structure 530 is connected to the second connecting structure to form an ester bond 522′.

The side-chain structure 540 is a fatty acid. The side-chain structure 540 is connected to the first connecting structure to form an amide bond 521′.

The polar functional group 560 is connected to the side-chain structure 530.

### 6th Embodiment

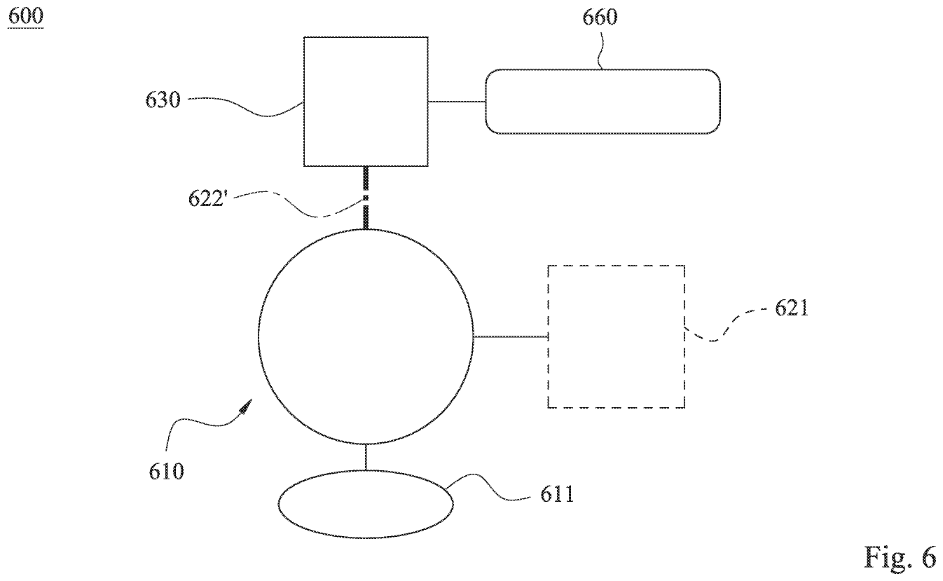

FIG. 6 is a structural schematic view of a molecule of a plasticizer 600 according to the 6th embodiment of the present disclosure. In FIG. 6, the molecule of the plasticizer 600 according to the 6th embodiment includes a central structure 610, two connecting structures, a side-chain structure 630 and a polar functional group 660.

The central structure 610 is an amino acid and includes a residue 611.

The two connecting structures are a first connecting structure and a second connecting structure and connected to the central structure 610, respectively. The first connecting structure is an amine group 621, and the second connecting structure is a carboxyl group (not shown in drawings).

The side-chain structure 630 is formed by polymerization with ethylene glycol or polyethylene glycol as monomers. The side-chain structure 630 is connected to the second connecting structure to form an ester bond 622′.

The polar functional group 660 is connected to the side-chain structure 630.

### 7th Embodiment

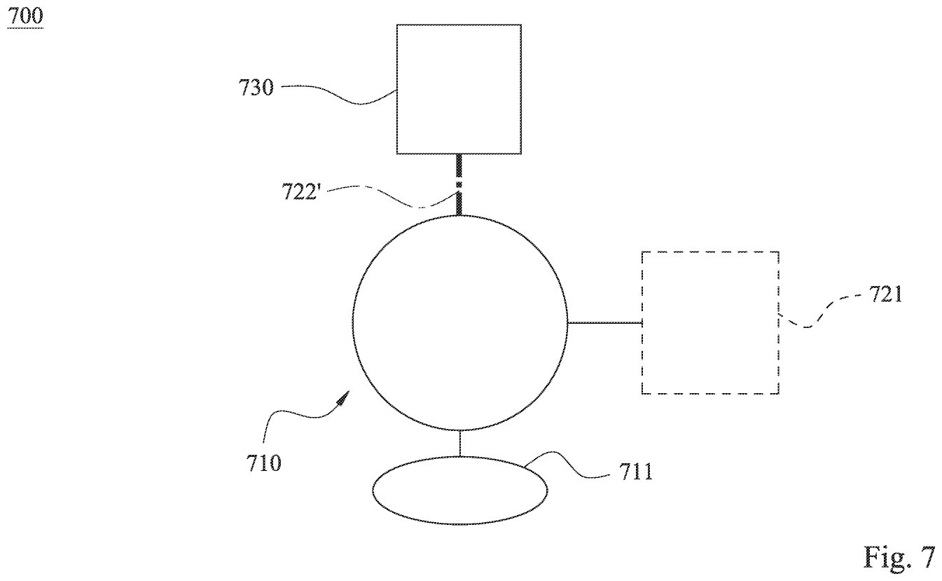

FIG. 7 is a structural schematic view of a molecule of a plasticizer 700 according to the 7th embodiment of the present disclosure. In FIG. 7, the molecule of the plasticizer 700 according to the 7th embodiment includes a central structure 710, two connecting structures and a side-chain structure 730.

The central structure 710 is an amino acid and includes a residue 711.

The two connecting structures are a first connecting structure and a second connecting structure and connected to the central structure 710, respectively. The first connecting structure is an amine group 721, and the second connecting structure is a carboxyl group (not shown in drawings).

The side-chain structure 730 is formed by polymerization with propylene glycol or polypropylene glycol as monomers. The side-chain structure 730 is connected to the second connecting structure to form an ester bond 722′.

### 8th Embodiment

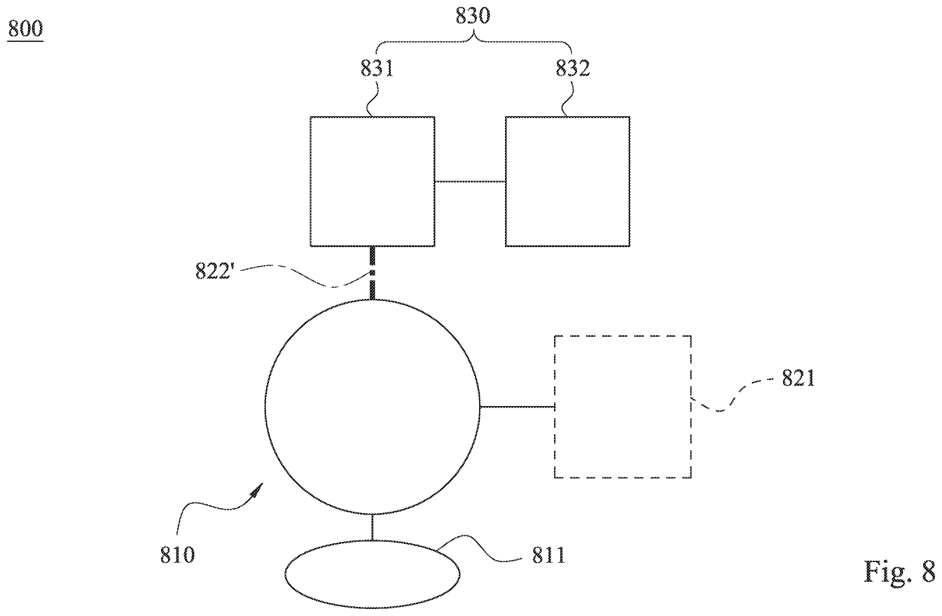

FIG. 8 is a structural schematic view of a molecule of a plasticizer 800 according to the 8th embodiment of the present disclosure. In FIG. 8, the molecule of the plasticizer 800 according to the 8th embodiment includes a central structure 810, two connecting structures and a side-chain structure 830.

The central structure 810 is an amino acid and includes a residue 811.

The two connecting structures are a first connecting structure and a second connecting structure and connected to the central structure 810, respectively. The first connecting structure is an amine group 821, and the second connecting structure is a carboxyl group (not shown in drawings).

The side-chain structure 830 is formed by a first short chain 831 connected to a second short chain 832. The first short chain 831 is formed by polymerization with ethylene glycol or polyethylene glycol as monomers. The second short chain 832 is formed by polymerization with propylene glycol or polypropylene glycol as monomers. The side-chain structure 830 is connected to the second connecting structure to form an ester bond 822′.

### 9th Embodiment

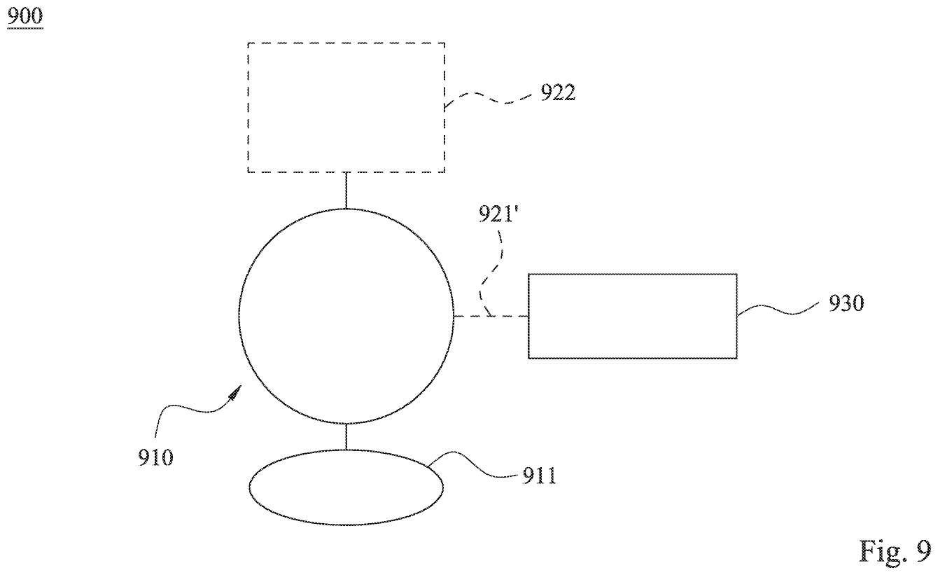

FIG. 9 is a structural schematic view of a molecule of a plasticizer 900 according to the 9th embodiment of the present disclosure. In FIG. 9, the molecule of the plasticizer 900 according to the 9th embodiment includes a central structure 910, two connecting structures and a side-chain structure 930.

The central structure 910 is an amino acid and includes a residue 911.

The two connecting structures are a first connecting structure and a second connecting structure and connected to the central structure 910, respectively. The first connecting structure is an amine group (not shown in drawings), and the second connecting structure is a carboxyl group 922.

The side-chain structure 930 is a fatty acid. The side-chain structure 930 is connected to the first connecting structure to form an amide bond 921.

### 10th Embodiment

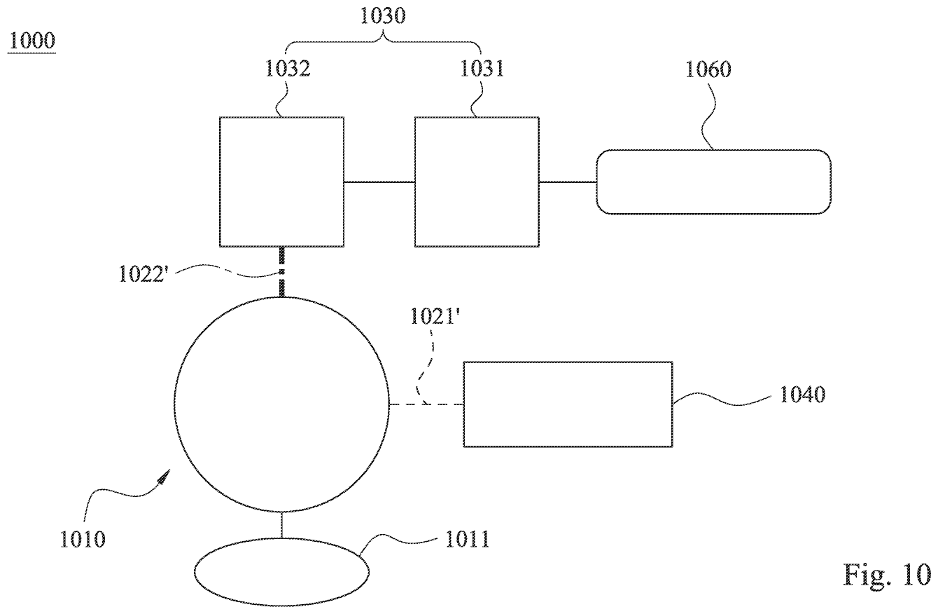

FIG. 10 is a structural schematic view of a molecule of a plasticizer 1000 according to the 10th embodiment of the present disclosure. In FIG. 10, the molecule of the plasticizer 1000 according to the 10th embodiment includes central structure 1010, two connecting structures, two side-chain structures 1030, 1040 and a polar functional group 1060.

The central structure 1010 is an amino acid and includes a residue 1011.

The two connecting structures are a first connecting structure and a second connecting structure and connected to the central structure 1010, respectively. The first connecting structure is an amine group (not shown in drawings), and the second connecting structure is a carboxyl group (not shown in drawings).

The side-chain structure 1030 is formed by a first short chain 1031 connected to a second short chain 1032. The first short chain 1031 is formed by polymerization with ethylene glycol or polyethylene glycol as monomers. The second short chain 1032 is formed by polymerization with propylene glycol or polypropylene glycol as monomers. The side-chain structure 1030 is connected to the second connecting structure to form an ester bond 1022′.

The side-chain structure 1040 is a fatty acid. The side-chain structure 1040 is connected to the first connecting structure to form an amide bond 1021′.

The polar functional group 1060 is connected to the side-chain structure 1030.

### 11th Embodiment

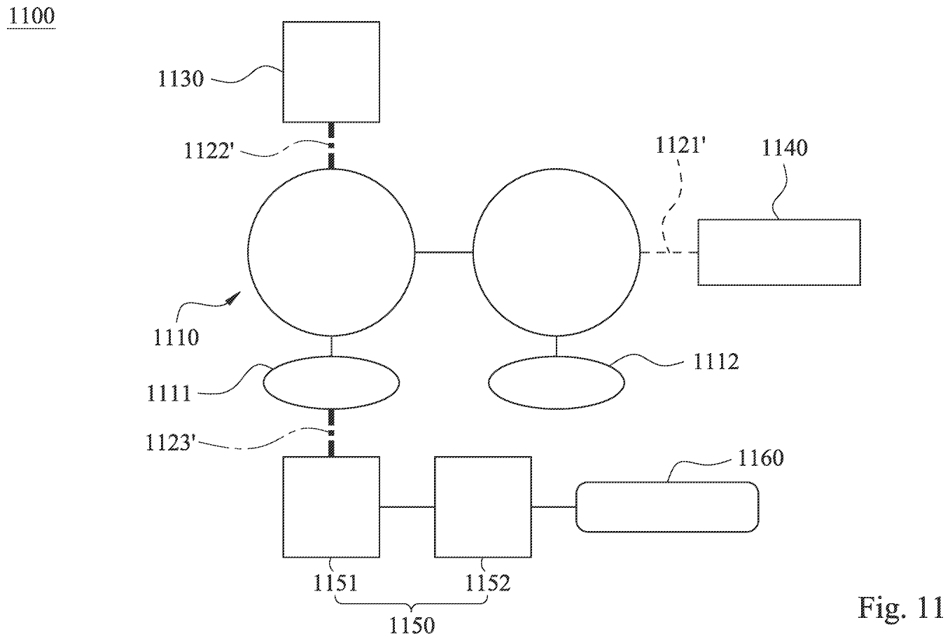

FIG. 11 is a structural schematic view of a molecule of a plasticizer 1100 according to the 11th embodiment of the present disclosure. In FIG. 11, the molecule of the plasticizer 1100 according to the 11th embodiment includes a central structure 1110, three connecting structures, three side-chain structures 1130, 1140, 1150 and a polar functional group 1160.

The central structure 1110 includes two amino acids, and each of the two amino acids includes a residue 1111, 1112.

The three connecting structures are a first connecting structure, a second connecting structure and a third connecting structure and connected to the central structure 1110, respectively. The first connecting structure is an amine group (not shown in drawings), and each of the second connecting structure and the third connecting structure is a carboxyl group (not shown in drawings). The third connecting structure is connected to the residue 1111.

The side-chain structure 1130 is formed by polymerization with ethylene glycol or polyethylene glycol as monomers. The side-chain structure 1130 is connected to the second connecting structure to form an ester bond 1122′.

The side-chain structure 1140 is a fatty acid. The side-chain structure 1140 is connected to the first connecting structure to form an amide bond 1121′.

The side-chain structure 1150 is formed by a first short chain 1151 connected to a second short chain 1152. The first short chain 1151 is formed by polymerization with ethylene glycol or polyethylene glycol as monomers. The second short chain 1152 is formed by polymerization with propylene glycol or polypropylene glycol as monomers. The side-chain structure 1150 is connected to the third connecting structure to form an ester bond 1123′.

The polar functional group 1160 is connected to the side-chain structure 1150.

### 12th Embodiment

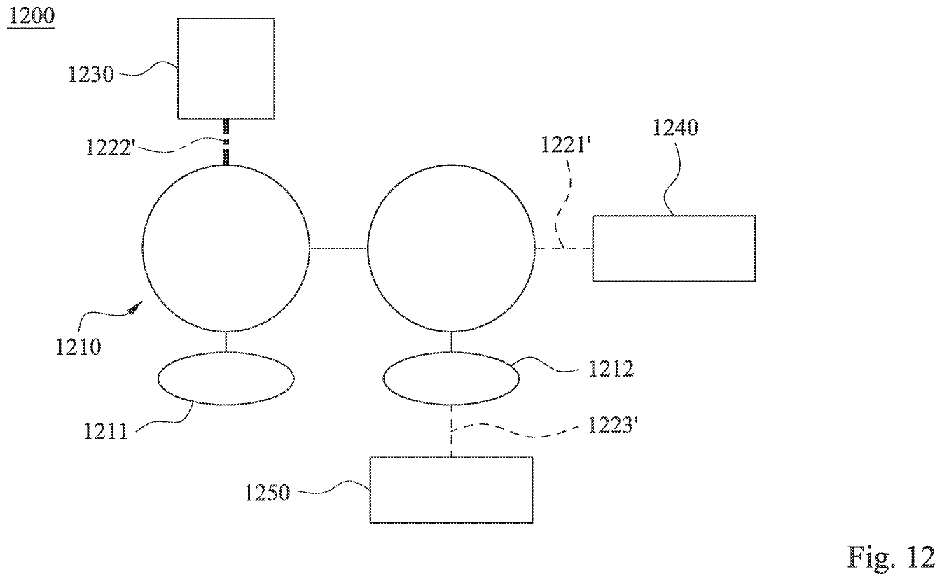

FIG. 12 is a structural schematic view of a molecule of a plasticizer 1200 according to the 12th embodiment of the present disclosure. In FIG. 12, the molecule of the plasticizer 1200 according to the 12th embodiment includes a central structure 1210, three connecting structures and three side-chain structures 1230, 1240, 1250.

The central structure 1210 includes two amino acids, and each of the two amino acids includes a residue 1211, 1212.

The three connecting structures are a first connecting structure, a second connecting structure and a third connecting structure and connected to the central structure 1210, respectively. Each of the first connecting structure and the third connecting structure is an amine group (not shown in drawings), and the second connecting structure is a carboxyl group (not shown in drawings). The third connecting structure is connected to the residue 1212.

The side-chain structure 1230 is formed by polymerization with ethylene glycol or polyethylene glycol as monomers. The side-chain structure 1230 is connected to the second connecting structure to form an ester bond 1222′.

The side-chain structure 1240 is a fatty acid. The side-chain structure 1240 is connected to the first connecting structure to form an amide bond 1221′.

The side-chain structure 1250 is a fatty acid. The side-chain structure 1250 is connected to the third connecting structure to form an amide bond 1223′.

### 13th Embodiment

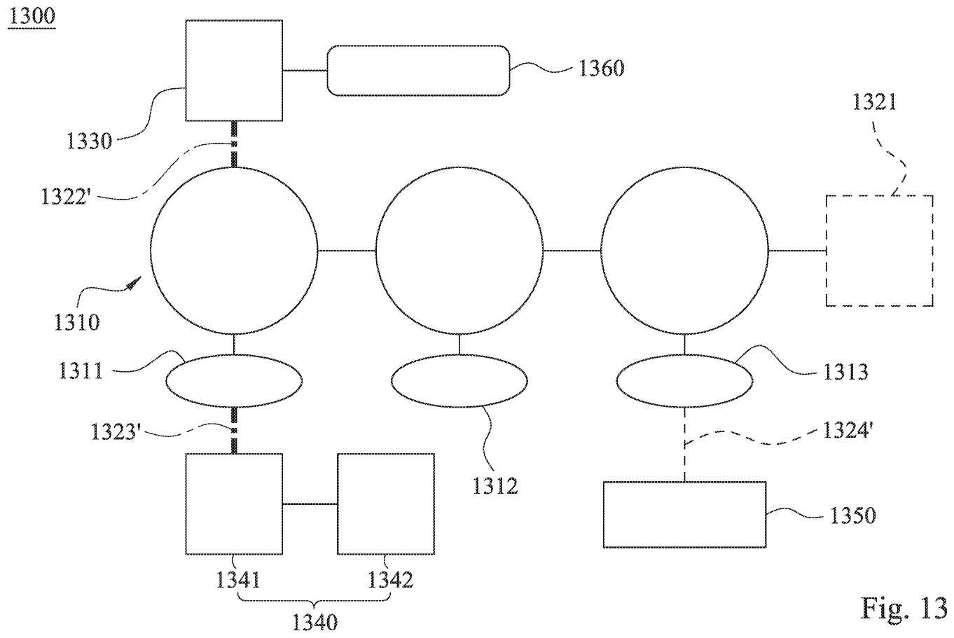

FIG. 13 is a structural schematic view of a molecule of a plasticizer 1300 according to the 13th embodiment of the present disclosure. In FIG. 13, the molecule of the plasticizer 1300 according to the 13th embodiment includes a central structure 1310, four connecting structures, three side-chain structures 1330, 1340, 1350 and a polar functional group 1360.

The central structure 1310 includes three amino acids, and each of the three amino acids includes a residue 1311, 1312, 1313.

The four connecting structures are a first connecting structure, a second connecting structure, a third connecting structure and a fourth connecting structure and connected to the central structure 1310, respectively. The first connecting structure is an amine group 1321, each of the second connecting structure and the third connecting structure is a carboxyl group (not shown in drawings), and the fourth connecting structure is an amine group (not shown in drawings). The third connecting structure and the fourth connecting structure are connected to the residue 1311 and the residue 1313, respectively.

The side-chain structure 1330 is formed by polymerization with ethylene glycol or polyethylene glycol as monomers. The side-chain structure 1330 is connected to the second connecting structure to form an ester bond 1322′.

The side-chain structure 1340 is formed by a first short chain 1341 connected to a second short chain 1342. The first short chain 1341 is formed by polymerization with ethylene glycol or polyethylene glycol as monomers. The second short chain 1342 is formed by polymerization with propylene glycol or polypropylene glycol as monomers. The side-chain structure 1340 is connected to the third connecting structure to form an ester bond 1323′.

The side-chain structure 1350 is a fatty acid. The side-chain structure 1350 is connected to the fourth connecting structure to form an amide bond 1324′.

The polar functional group 1360 is connected to the side-chain structure 1330.

### 14th Embodiment

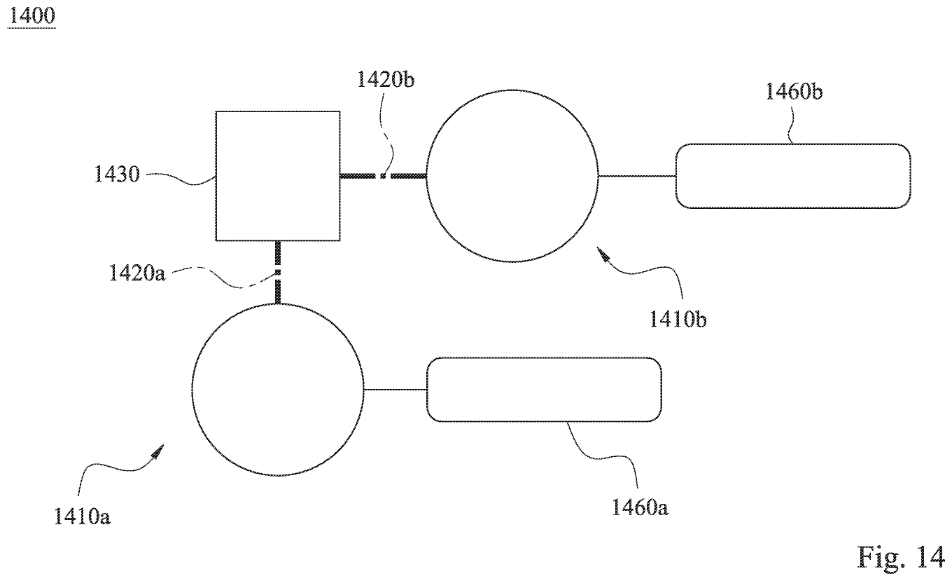

FIG. 14 is a structural schematic view of a molecule of a plasticizer 1400 according to the 14th embodiment of the present disclosure. In FIG. 14, the molecule of the plasticizer 1400 according to the 14th embodiment includes two central structures 1410a, 1410b, two connecting structures, a side-chain structure 1430 and two polar functional groups 1460a, 1460b.

Each of the central structures 1410a, 1410b is a benzene derivative.

Each of the central structures 1410a, 1410b is connected to the corresponding one of the connecting structures, that is, the central structure 1410a is connected to one of the connecting structures, and the central structure 1410b is connected to the other one of the connecting structures. Both of the connecting structures are carboxyl groups (not shown in drawings).

The side-chain structure 1430 is formed by polymerization with ethylene glycol or polyethylene glycol as monomers. Each of the central structures 1410a, 1410b is connected to the side-chain structure 1430 through the connecting structure to form an ester bond 1420a, 1420b.

Each of the polar functional groups 1460a, 1460b is connected to the corresponding one of the central structures 1410a, 1410b.

According to the above description of the present disclosure, the following specific examples are provided for further explanation.

In the 1st Example to the 8th Example, the side-chain structures connected to the amine group, the carboxyl group and the hydroxyl group are named as SCA, SCC and SCH, respectively. For the molecule of the plasticizer including a plurality of the side-chain structures connected to a same functional group, for example, two side-chain structures are connected to the amine group in the 1st Example, so the two side-chain structures are named as SCA1 and SCA2, respectively. The names of other side-chain structures follow the same rule, and an explanation in this regard will not be provided again.

| | 1st Example | 2nd Example |
|---|---|---|
| Central Structure | Aminobenzoic Acid | Aminobenzoic Acid |
| Structure of Para Position | 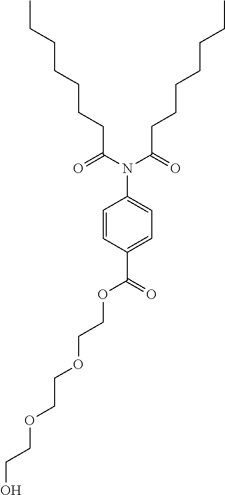 [SMILES: CCCCCCCC(=O)N(C(=O)CCCCCCC)c1ccc(C(=O)OCCOCCOCCO)cc1] | 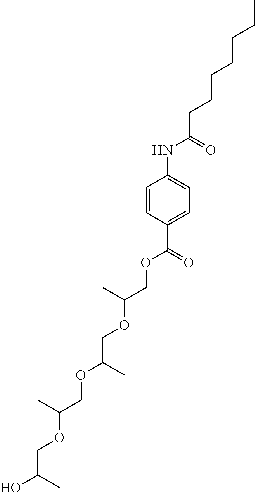 [SMILES: CCCCCCCC(=O)Nc1ccc(C(=O)OCC(C)OCC(C)OCC(C)OCC(C)O)cc1] |
| SMILES Notation | C1═CC(═CC═C1C(═O)OC | C1═CC(═CC═C1C(═O)OC |
| | COCCOCCO[H])N(C(CCC | C(OCC(OCC(OCC(O[H])C) |
| | CCCC)═O)C(CCCCCCC)═O | C)C)C)N(C(CCCCCCC)═O)[H] |
| InChI Notation | InChI = 1S/C29H47NO7/c1- | InChI = 1S/C27H45NO7/c1- |
| | 3-5-7-9-11-13-27(32)30(28 | 6-7-8-9-10-11-26(30)28-25- |
| | (33)14-12-10-8-6-4-2)26-17- | 14-12-24(13-15-25)27(31) |
| | 15-25(16-18-26)29(34)37- | 35-19-23(5)34-18-22(4)33- |
| | 24-23-36-22-21-35-20-19- | 17-21(3)32-16-20(2)29/h12- |
| | 31/h15-18, 31H, 3-14, 19- | 15, 20-23, 29H, 6-11, 16-1 |
| | 24H2, 1-2H3 | 9H2, 1-5H3, (H, 28, 30) |
| Molecular Formula | C₂₉H₄₇NO₇ | C₂₇H₄₅NO₇ |
| Molecular Weight | 521.693 | 495.655 |
| MwP | | |
| (g/mol, Dalton) | | |
| Structure of Ortho Position | With Steric Hindrance | 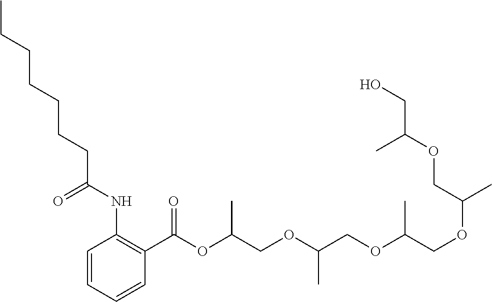 [SMILES: CCCCCCCC(=O)Nc1ccccc1C(=O)OC(C)COC(C)COC(C)COC(C)COC(C)CO] |
| Structure of Meta Position | 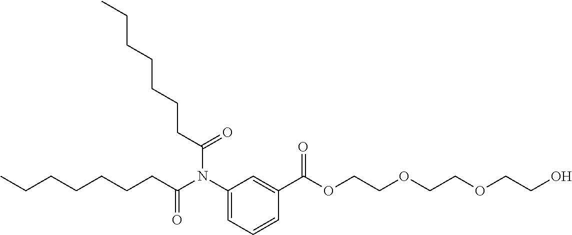 [SMILES: CCCCCCCC(=O)N(C(=O)CCCCCCC)c1cccc(C(=O)OCCOCCOCCO)c1] | 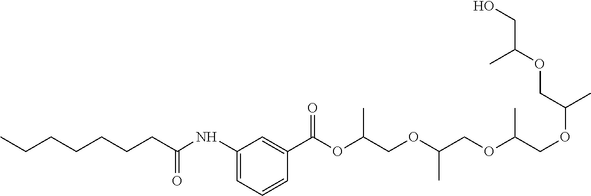 [SMILES: CCCCCCCC(=O)Nc1cccc(C(=O)OC(C)COC(C)COC(C)COC(C)COC(C)CO)c1] |

Side-Chain Structure SCA1 Connected to Amine Group

| | | |
|---|---|---|
| nNC | 8 | 8 |
| nNO | 0 | 0 |
| nNC/(nNC + nNO) | 1 | 1 |
| Polar Functional | — | — |
| Group | | |

Side-Chain Structure SCA2 Connected to Amine Group

| | | |
|---|---|---|
| nNC | 8 | 0 |
| nNO | 0 | 0 |
| nNC/(nNC + nNO) | 1 | — |
| Polar Functional | — | — |
| Group | | |

Side-Chain Structure SCC Connected to Carboxyl Group

| | | |
|---|---|---|
| nOC | 6 | 12 |
| nOO | 2 | 3 |
| nOC/(nOC + nOO) | 0.75 | 0.80 |
| Polar Functional | OH | OH |
| Group | | |

Relationship Between Numbers of Atoms of
Side-Chain Structures SCA1, SCA2 and SCC

| | | |
|---|---|---|
| nNC_{SCA1}/(nNC_{SCA1} + | 0.6 | 0.4 |
| nOC_{SCC}) | | |
| nNC_{SCA2}/(nNC_{SCA2} + | 0.6 | 0.0 |
| nOC_{SCC}) | | |
| nNC_{SCA1}/(nNC_{SCA1} + | 0.8 | 0.7 |
| nOO_{SCC}) | | |
| nNC_{SCA2}/(nNC_{SCA2} + | 0.8 | 0.0 |
| nOO_{SCC}) | | |
| nNO_{SCA1}/(nNO_{SCA1} + | 0.0 | 0.0 |
| nOC_{SCC}) | | |
| nNO_{SCA2}/(nNO_{SCA2} + | 0.0 | 0.0 |
| nOC_{SCC}) | | |
| nNO_{SCA1}/(nNO_{SCA1} + | 0.0 | 0.0 |
| nOO_{SCC}) | | |
| nNO_{SCA2}/(nNO_{SCA2} + | 0.0 | 0.0 |
| nOO_{SCC}) | | |

Furthermore, in the 1st Example to the 8th Example, a number of carbon atoms and a number of oxygen atoms of the side-chain structure SCA1 are nNC_{SCA1} and nNO_{SCA1}, respectively. A number of carbon atoms and a number of oxygen atoms of the side-chain structure SCA2 are nNC_{SCA2} and nNO_{SCA2}, respectively. A number of carbon atoms and a number of oxygen atoms of the side-chain structure SCC are nOC_{SCC} and nOO_{SCC}, respectively. SCA1, SCA2 and SCC are only for marking the side-chain structures to clearly understand the characteristics of the side-chain structures, and do not make a difference to the calculation of carbon atoms and oxygen atoms of nNO, nNC, nOC and nOO as claimed. That is, nNC_{SCA1} is taken as nNC, nNO_{SCA1} is taken nNO, nNC_{SCA2} is taken as nNC, nNO_{SCA2} is taken as nNO, nOC_{SCC} is taken as nOC and nOO_{SCC} is taken as nOO during calculation. The information in the following tables has the same definition, and an explanation in this regard will not be provided again.

| | 3rd Example | 4th Example |
|---|---|---|
| Central Structure | Aminobenzoic Acid | Hydroxybenzoic Acid |
| Structure of Para Position | 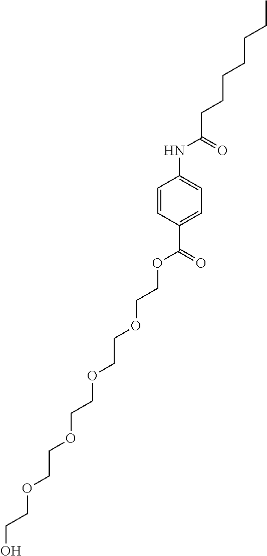 [SMILES: CCCCCCCC(=O)Nc1ccc(C(=O)OCCOCCOCCOCCOCCO)cc1] | 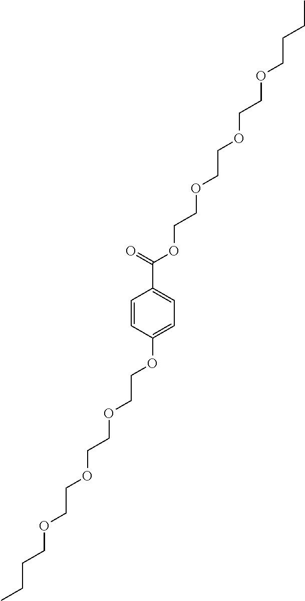 [SMILES: CCCCOCCOCCOCCOC(=O)c1ccc(OCCOCCOCCOCCCC)cc1] |
| SMILES Notation | C1═CC(═CC═C1C(═O)OC | C1═CC(═CC═C1C(═O)OC |
| | COCCOCCOCCOCCO[H]) | COCCOCCOCCCC)OCC |
| | N(C(CCCCCCC)═O)[H] | OCCOCCOCCCC |
| InChI Notation | InChI = 1S/C25H41NO8/c1- | InChI = 1S/C27H46O9/c1-3- |
| | 2-3-4-5-6-7-24(28)26-23-10- | 5-11-29-13-15-31-17-19-33- |
| | 8-22(9-11-23)25(29)34-21- | 21-23-35-26-9-7-25(8-10- |
| | 20-33-19-18-32-17-16-31- | 26)27(28)36-24-22-34-20- |
| | 15-14-30-13-12-27/h8-11, | 18-32-16-14-30-12-6-4-2/h |
| | 27H, 2-7, 12-21H2, 1H3, (H, | 7-10H, 3-6, 11-24H2, 1-2H3 |
| | 26, 28) | |
| Molecular Formula | C₂₅H₄₁NO₈ | C₂₇H₄₆O₉ |
| Molecular Weight | 483.601 | 514.655 |
| MwP | | |
| (g/mol, Dalton) | | |
| Structure of Ortho Position | 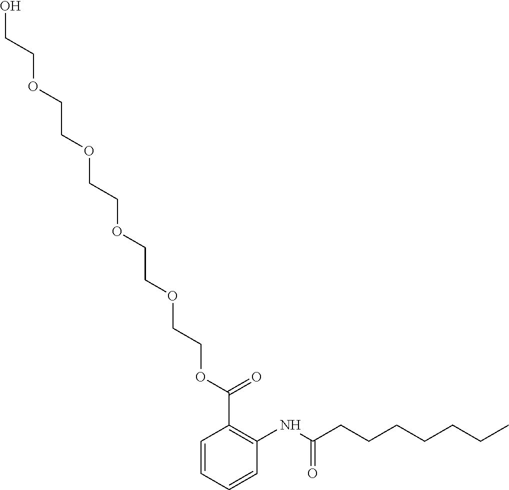 [SMILES: CCCCCCCC(=O)Nc1ccccc1C(=O)OCCOCCOCCOCCOCCO] | 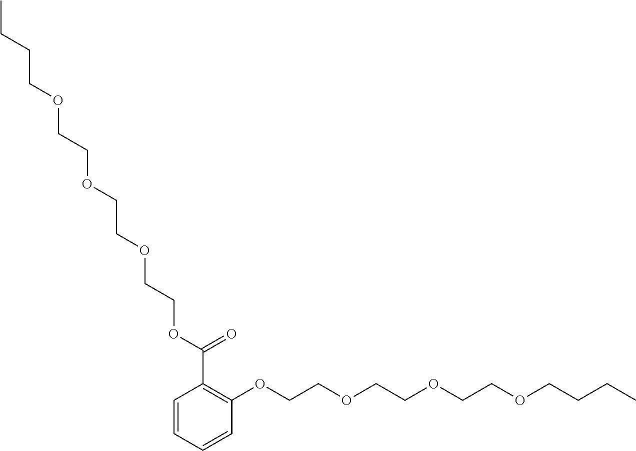 [SMILES: CCCCOCCOCCOCCOC(=O)c1ccccc1OCCOCCOCCOCCCC] |
| Structure of Meta Position | 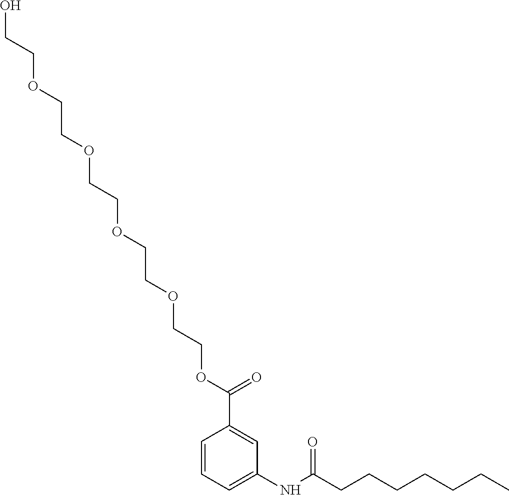 [SMILES: CCCCCCCC(=O)Nc1cccc(C(=O)OCCOCCOCCOCCOCCO)c1] | 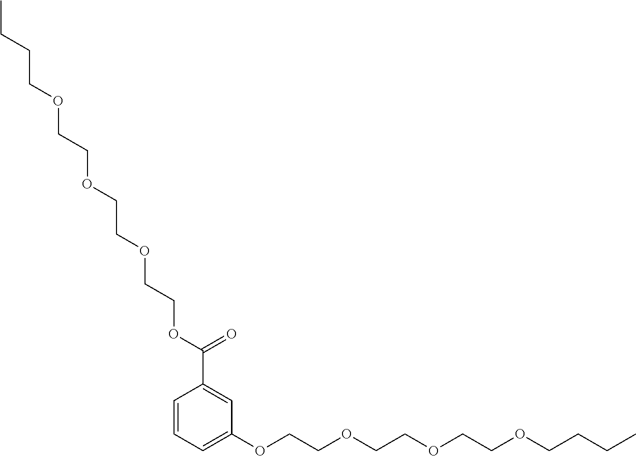 [SMILES: CCCCOCCOCCOCCOC(=O)c1cccc(OCCOCCOCCOCCCC)c1] |

Side-Chain Structure SCA1 Connected to Amine Group

| | | |
|---|---|---|
| nNC | 8 | 0 |
| nNO | 0 | 0 |
| nNC/(nNC + nNO) | 1 | — |
| Polar Functional | — | — |
| Group | | |

Side-Chain Structure SCA2 Connected to Amine Group

| | | |
|---|---|---|
| nNC | 0 | 0 |
| nNO | 0 | 0 |
| nNC/(nNC + nNO) | — | — |
| Polar Functional | — | — |
| Group | | |

Side-Chain Structure SCC Connected to Carboxyl Group

| | | |
|---|---|---|
| nOC | 10 | 10 |
| nOO | 4 | 3 |
| nOC/(nOC + nOO) | 0.71 | 0.77 |
| Polar Functional | OH | — |
| Group | | |

Relationship Between Numbers of Atoms of
Side-Chain Structures SCA1, SCA2 and SCC

| | | |
|---|---|---|
| nNC_{SCA1}/(nNC_{SCA1} + | 0.4 | 0.0 |
| nOC_{SCC}) | | |
| nNC_{SCA2}/(nNC_{SCA2} + | 0.0 | 0.0 |
| nOC_{SCC}) | | |
| nNC_{SCA1}/(nNC_{SCA1} + | 0.7 | 0.0 |
| nOO_{SCC}) | | |
| nNC_{SCA2}/(nNC_{SCA2} + | 0.0 | 0.0 |
| nOO_{SCC}) | | |
| nNO_{SCA1}/(nNO_{SCA1} + | 0.0 | 0.0 |
| nOC_{SCC}) | | |
| nNO_{SCA2}/(nNO_{SCA2} + | 0.0 | 0.0 |
| nOC_{SCC}) | | |
| nNO_{SCA1}/(nNO_{SCA1} + | 0.0 | 0.0 |
| nOO_{SCC}) | | |
| nNO_{SCA2}/(nNO_{SCA2} + | 0.0 | 0.0 |
| nOO_{SCC}) | | |

Side-Chain Structure SCH1 Connected to Hydroxyl Group

| | | |
|---|---|---|
| nO′C | — | 10 |
| nO′O | — | 3 |
| nO′C/(nO′C + nO′O) | — | 0.77 |
| Polar Functional | — | — |
| Group | | |

Side-Chain Structure SCH2 Connected to Hydroxyl Group

| | | |
|---|---|---|
| nO′C | — | 0 |
| nO′O | — | 0 |
| nO′C/(nO′C + nO′O) | — | — |
| Polar Functional | — | — |
| Group | | |

Side-Chain Structure SCH3 Connected to Hydroxyl Group

| | | |
|---|---|---|
| nO′C | — | 0 |
| nO′O | — | 0 |
| nO′C/(nO′C + nO′O) | — | — |
| Polar Functional | — | — |
| Group | | |

| | 5th Example | 6th Example |
|---|---|---|
| Central Structure | Hydroxybenzoic Acid | Propane-1,2,3-Triol |
| Structure of Para Position | 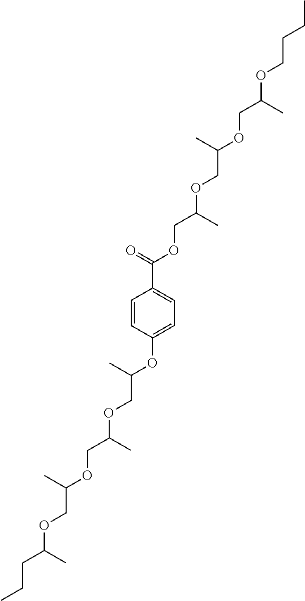 [SMILES: CCCCOC(C)COC(C)COC(C)COC(=O)c1ccc(OC(C)COC(C)COC(C)COC(C)CCC)cc1] | 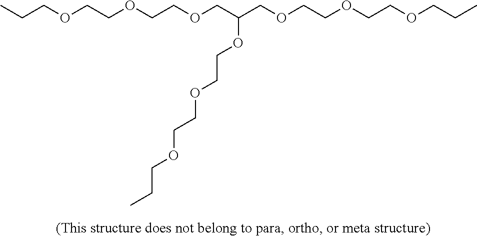 [SMILES: CCCOCCOCCOCC(COCCOCCOCCC)OCCOCCOCCC] |
| SMILES Notation | C1═CC(═CC═C1C(═O)OC | C(C(COCCOCCOCCC)O |
| | C(OCC(OCC(OCCCC)C)C) | CCOCCOCCC)OCCOCC |
| | C)OC(COC(COC(COC(C | OCCC |
| | CC)C)C)C)C | |
| InChI Notation | InChI = 1S/C34H60O9/c1-10- | InChI = 1S/C24H50O9/c1-4- |
| | 12-18-36-26(4)19-38-28 | 7-25-10-13-28-16-18-31-22- |
| | (6)21-40-30(8)23-42-34(35) | 24(33-21-20-30-15-12-27- |
| | 32-14-16-33(17-15-32)43- | 9-6-3)23-32-19-17-29-14- |
| | 31(9)24-41-29(7)22-39-27 | 11-26-8-5-2/h24H, 4-23H2, |
| | (5)20-37-25(3)13-11-2/h14- | 1-3H3 |
| | 17, 25-31H, 10-13, 18-24H2, | |
| | 1-9H3 | |
| Molecular Formula | C₃₄H₆₀O₉ | C₂₄H₅₀O₉ |
| Molecular Weight | 612.843 | 482.654 |
| MwP | | |
| (g/mol, Dalton) | | |
| Structure of Ortho Position | 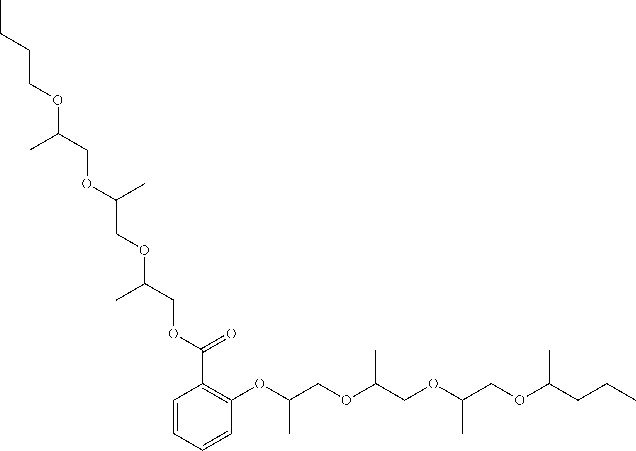 [SMILES: CCCCOC(C)COC(C)COC(C)COC(=O)c1ccccc1OC(C)COC(C)COC(C)COC(C)CCC] | — |
| Structure of Meta Position | 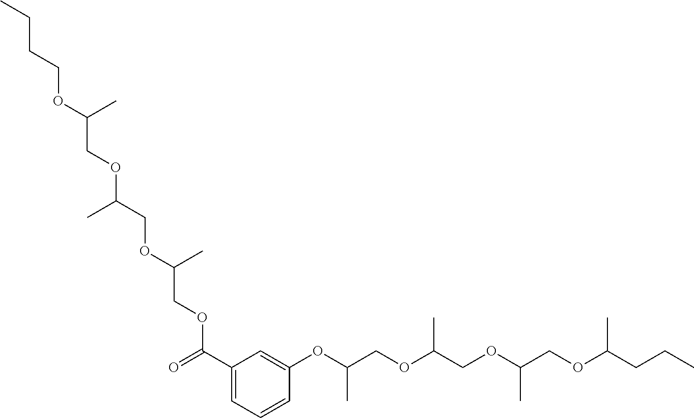 [SMILES: CCCCOC(C)COC(C)COC(C)COC(=O)c1cccc(OC(C)COC(C)COC(C)COC(C)CCC)c1] | — |

Side-Chain Structure SCA1 Connected to Amine Group

| | | |
|---|---|---|
| nNC | 0 | 0 |
| nNO | 0 | 0 |
| nNC/(nNC + nNO) | — | — |
| Polar Functional | — | — |
| Group | | |

Side-Chain Structure SCA2 Connected to Amine Group

| | | |
|---|---|---|
| nNC | 0 | 0 |
| nNO | 0 | 0 |
| nNC/(nNC + nNO) | — | — |
| Polar Functional | — | — |
| Group | | |

Side-Chain Structure SCC Connected to Carboxyl Group

| | | |
|---|---|---|
| nOC | 13 | 0 |
| nOO | 3 | 0 |
| nOC/(nOC + nOO) | 0.81 | — |
| Polar Functional | — | — |
| Group | | |

Relationship Between Numbers of Atoms of
Side-Chain Structures SCA1, SCA2 and SCC

| | | |
|---|---|---|
| nNC_{SCA1}/(nNC_{SCA1} + | 0.0 | — |
| nOC_{SCC}) | | |
| nNC_{SCA2}/(nNC_{SCA2} + | 0.0 | — |
| nOC_{SCC}) | | |
| nNC_{SCA1}/(nNC_{SCA1} + | 0.0 | — |
| nOO_{SCC}) | | |
| nNC_{SCA2}/(nNC_{SCA2} + | 0.0 | — |
| nOO_{SCC}) | | |
| nNO_{SCA1}/(nNO_{SCA1} + | 0.0 | — |
| nOC_{SCC}) | | |
| nNO_{SCA2}/(nNO_{SCA2} + | 0.0 | — |
| nOC_{SCC}) | | |
| nNO_{SCA1}/(nNO_{SCA1} + | 0.0 | — |
| nOO_{SCC}) | | |
| nNO_{SCA2}/(nNO_{SCA2} + | 0.0 | — |
| nOO_{SCC}) | | |

Side-Chain Structure SCH1 Connected to Hydroxyl Group

| | | |
|---|---|---|
| nO′C | 14 | 7 |
| nO′O | 3 | 2 |
| nO′C/(nO′C + nO′O) | 0.82 | 0.78 |
| Polar Functional | — | — |
| Group | | |

Side-Chain Structure SCH2 Connected to Hydroxyl Group

| | | |
|---|---|---|
| nO′C | 0 | 7 |
| nO′O | 0 | 2 |
| nO′C/(nO′C + NO′O) | — | 0.78 |
| Polar Functional | — | — |
| Group | | |

Side-Chain Structure SCH3 Connected to Hydroxyl Group

| | | |
|---|---|---|
| nO′C | 0 | 7 |
| nO′O | 0 | 2 |
| nO′C/(nO′C + nO′O) | — | 0.78 |
| Polar Functional | — | — |
| Group | | |

| | 7th Example | 8th Example |
|---|---|---|
| Central Structure | Propane-1,2,3-Triol | 2-(2-Hydroxyethoxy) |
| | | Propan-1-ol |
| Structure | 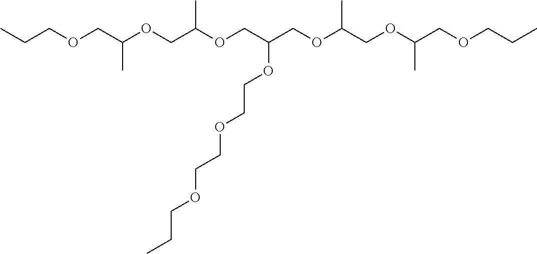 [SMILES: CCCOCCOCCOC(COC(C)COC(C)COCCC)COC(C)COC(C)COCCC] | 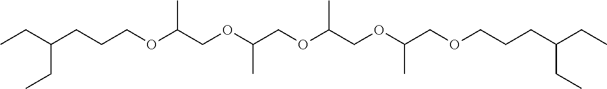 [SMILES: CCC(CC)CCCOCC(C)OCC(C)OCC(C)OCC(C)OCCCC(CC)CC] |
| SMILES Notation | C(C(COC(COC(COCCC)C) | C(OC(COC(COCCCC(CC) |
| | C)OCCOCCOCCC)OC(C | CC)C)C)C(OCC(OCCCC |
| | OC(COCCC)C)C | (CC)CC)C)C |
| InChI Notation | InChI = 1S/C28H58O9/c1-8- | InChI = 1S/C28H58O5/c1-9- |
| | 11-29-14-15-30-16-17-33- | 27(10-2)15-13-17-29-19-23 |
| | 28(22-36-26(6)20-34-24(4) | (5)31-21-25(7)33-22-26(8) |
| | 18-31-12-9-2)23-37-27(7)21- | 32-20-24(6)30-18-14-16-28 |
| | 35-25(5)19-32-13-10-3/h | (11-3)12-4/h23-28H, 9-22 |
| | 24-28H, 8-23H2, 1-7H3 | H2, 1-8H3 |
| Molecular Formula | C₂₈H₅₈O₉ | C₂₈H₅₈O₅ |
| Molecular Weight | 538.761 | 474.763 |
| MwP | | |
| (g/mol, Dalton) | | |

Side-Chain Structure SCA1 Connected to Amine Group

| | | |
|---|---|---|
| nNC | 0 | 0 |
| nNO | 0 | 0 |
| nNC/(nNC + nNO) | — | — |
| Polar Functional | — | — |
| Group | | |

Side-Chain Structure SCA2 Connected to Amine Group

| | | |
|---|---|---|
| nNC | 0 | 0 |
| nNO | 0 | 0 |
| nNC/(nNC + nNO) | — | — |
| Polar Functional | — | — |
| Group | | |

Side-Chain Structure SCC Connected to Carboxyl Group

| | | |
|---|---|---|
| nOC | 0 | 0 |
| nOO | 0 | 0 |
| nOC/(nOC + nOO) | — | — |
| Polar Functional | — | — |
| Group | | |

Relationship Between Numbers of Atoms of
Side-Chain Structures SCA1, SCA2 and SCC

| | | |
|---|---|---|
| nNC_{SCA1}/(nNC_{SCA1} + | — | — |
| nOC_{SCC}) | | |
| nNC_{SCA2}/(nNC_{SCA2} + | — | — |
| nOC_{SCC}) | | |
| nNC_{SCA1}/(nNC_{SCA1} + | — | — |
| nOO_{SCC}) | | |
| nNC_{SCA2}/(nNC_{SCA2} + | — | — |
| nOO_{SCC}) | | |
| nNO_{SCA1}/(nNO_{SCA1} + | — | — |
| nOC_{SCC}) | | |
| nNO_{SCA2}/(nNO_{SCA2} + | — | — |
| nOC_{SCC}) | | |
| nNO_{SCA1}/(nNO_{SCA1} + | — | — |
| nOO_{SCC}) | | |
| nNO_{SCA2}/(nNO_{SCA2} + | — | — |
| nOO_{SCC}) | | |

Side-Chain Structure SCH1 Connected to Hydroxyl Group

| | | |
|---|---|---|
| nO′C | 9 | 11 |
| nO′O | 2 | 1 |
| nO′C/(nO′C + nO′O) | 0.82 | 0.92 |
| Polar Functional | — | — |
| Group | | |

Side-Chain Structure SCH2 Connected to Hydroxyl Group

| | | |
|---|---|---|
| nO′C | 7 | 11 |
| nO′O | 2 | 1 |
| nO′C/(nO′C + nO′O) | 0.78 | 0.92 |
| Polar Functional | — | — |
| Group | | |

Side-Chain Structure SCH3 Connected to Hydroxyl Group

| | | |
|---|---|---|
| nO′C | 9 | 0 |
| nO′O | 2 | 0 |
| nO′C/(nO′C + nO′O) | 0.82 | — |
| Polar Functional | — | — |
| Group | | |

| | 9th Example | 10th Example | 11th Example |
|---|---|---|---|
| IUPAC Name of | Glycine | Alanine | Valine |
| Central Structure | | | |
| Structural Formula | 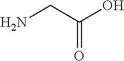 | 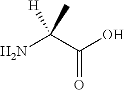 | 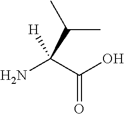 |
| Molecular Weight | 75.07 | 89.09 | 117.15 |
| MwP (g/mol) | | | |
| Abbreviation 1 | Gly | Ala | Val |
| Abbreviation 2 | G | A | V |
| Residue Property | Hydrophilic | Hydrophobic | Hydrophobic |
| Representative Structure as Side-Chain Structure Being PEG | 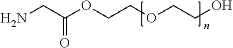 | 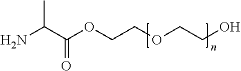 | 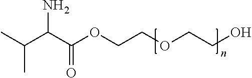 |
| Representative Structure as Side-Chain Structure Being PPG | 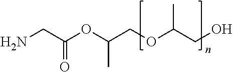 | 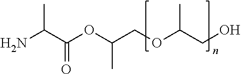 | 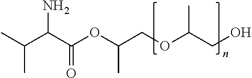 |
| Representative Structure as Side-Chain Structure Being Fatty Acid (CH₃(CH₂)ₙCOOH, n ≥ 3) | 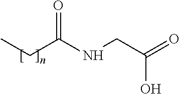 | 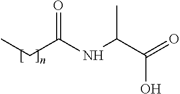 | 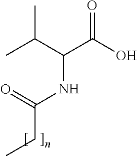 |

Side-Chain Structure Being PEG and PPG

| | | | |
|---|---|---|---|
| Number of | 12 | 12 | 12 |
| PEG Monomers | | | |
| Number of | 0 | 0 | 0 |
| PPG Monomers | | | |
| Molecular | | | |
| Weight, MwP | 603.7 | 617.7 | 645.8 |
| (g/mol, Dalton) | | | |
| nNC | 0 | 0 | 0 |
| nOC | 24 | 24 | 24 |
| nNO | 0 | 0 | 0 |
| nOO | 12 | 12 | 12 |
| nNC/(nNC + nOC) | 0 | 0 | 0 |
| nOC/nOO | 2 | 2 | 2 |
| nNO/(nNO + nOC) | 0 | 0 | 0 |
| nNO/(nNO + nOO) | 0 | 0 | 0 |
| Number of | 0 | 0 | 0 |
| PEG Monomers | | | |
| Number of | 12 | 12 | 12 |
| PPG Monomers | | | |
| Molecular | 772.0 | 786.0 | 814.1 |
| Weight, MwP | | | |
| (g/mol, Dalton) | | | |
| nNC | 0 | 0 | 0 |
| nOC | 36 | 36 | 36 |
| nNO | 0 | 0 | 0 |
| nOO | 12 | 12 | 12 |
| nNC/(nNC + nOC) | 0 | 0 | 0 |
| nOC/nOO | 3 | 3 | 3 |
| nNO/(nNO + nOC) | 0 | 0 | 0 |
| nNO/(nNO + nOO) | 0 | 0 | 0 |
| Number of | 6 | 6 | 6 |
| PEG Monomers | | | |
| Number of | 6 | 6 | 6 |
| PPG Monomers | | | |
| Molecular | 687.9 | 701.9 | 729.9 |
| Weight, MwP | | | |
| (g/mol, Dalton) | | | |
| nNC | 0 | 0 | 0 |
| nOC | 30 | 30 | 30 |
| nNO | 0 | 0 | 0 |
| nOO | 12 | 12 | 12 |
| nNC/(nNC + nOC) | 0 | 0 | 0 |
| nOC/nOO | 3 | 3 | 3 |
| nNO/(nNO + nOC) | 0 | 0 | 0 |
| nNO/(nNO + nOO) | 0 | 0 | 0 |
| Number of | 2 | 2 | 2 |
| PEG Monomers | | | |
| Number of | 5 | 5 | 5 |
| PPG Monomers | | | |
| Molecular | 453.6 | 467.6 | 495.7 |
| Weight, MwP | | | |
| (g/mol, Dalton) | | | |
| nNC | 0 | 0 | 0 |
| nOC | 19 | 19 | 19 |
| nNO | 0 | 0 | 0 |
| nOO | 7 | 7 | 7 |
| nNC/(nNC + nOC) | 0 | 0 | 0 |
| nOC/nOO | 3 | 3 | 3 |
| nNO/(nNO + nOC) | 0 | 0 | 0 |
| nNO/(nNO + nOO) | 0 | 0 | 0 |
| Number of | 5 | 5 | 5 |
| PEG Monomers | | | |
| Number of | 2 | 2 | 2 |
| PPG Monomers | | | |
| Molecular | 411.5 | 425.5 | 453.6 |
| Weight, MwP | | | |
| (g/mol, Dalton) | | | |
| nNC | 0 | 0 | 0 |
| nOC | 16 | 16 | 16 |
| nNO | 0 | 0 | 0. |
| nOO | 7 | 7 | 7 |
| nNC/(nNC + nOC) | 0 | 0 | 0 |
| nOC/nOO | 2 | 2 | 2 |
| nNO/(nNO + nOC) | 0 | 0 | 0 |
| nNO/(nNO + nOO) | 0 | 0 | 0 |
| Number of | 5 | 5 | 5 |
| PEG Monomers | | | |
| Number of | 5 | 5 | 5 |
| PPG Monomers | | | |
| Molecular | 585.7 | 599.8 | 627.8 |
| Weight, MwP | | | |
| (g/mol, Dalton) | | | |
| nNC | 0 | 0 | 0 |
| nOC | 25 | 25 | 25 |
| nNO | 0 | 0 | 0 |
| nOO | 10 | 10 | 10 |
| nNC/(nNC + nOC) | 0 | 0 | 0 |
| nOC/nOO | 3 | 3 | 3 |
| nNO/(nNO + nOC) | 0 | 0 | 0 |
| nNO/(nNO + nOO) | 0 | 0 | 0 |
| Number of | 4 | 4 | 4 |
| PEG Monomers | | | |
| Number of | 4 | 4 | 4 |
| PPG Monomers | | | |
| Molecular | 483.6 | 497.6 | 525.7 |
| Weight, MwP | | | |
| (g/mol, Dalton) | | | |
| nNC | 0 | 0 | 0 |
| nOC | 20 | 20 | 20 |
| nNO | 0 | 0 | 0 |
| nOO | 8 | 8 | 8 |
| nNC/(nNC + nOC) | 0 | 0 | 0 |
| nOC/nOO | 3 | 3 | 3 |
| nNO/(nNO + nOC) | 0 | 0 | 0 |
| nNO/(nNO + nOO) | 0 | 0 | 0 |
| Number of | 8 | 8 | 8 |
| PEG Monomers | | | |
| Number of | 8 | 8 | 8 |
| PPG Monomers | | | |
| Molecular | 892.1 | 906.1 | 934.2 |
| Weight MwP | | | |
| (g/mol, Dalton) | | | |
| nNC | 0 | 0 | 0 |
| nOC | 40 | 40 | 40 |
| nNO | 0 | 0 | 0 |
| nOO | 16 | 16 | 16 |
| nNC/(nNC + nOC) | 0 | 0 | 0 |
| nOC/nOO | 3 | 3 | 3 |
| nNO/(nNO + nOC) | 0 | 0 | 0 |
| nNO/(nNO + nOO) | 0 | 0 | 0 |

Side-Chain Structure Being PEG, PPG and Fatty Acid

| | | | |
|---|---|---|---|
| Number of | 3 | 3 | 3 |
| PEG Monomers | | | |
| Number of | 3 | 3 | 3 |
| PPG Monomers | | | |
| Fatty Acid | 3 | 3 | 3 |
| CH₃(CH₂)ₙCOOH, | | | |
| n = ? | | | |
| Molecular | 465.5 | 479.5 | 507.6 |
| Weight, MwP | | | |
| (g/mol, Dalton) | | | |
| nNC | 5 | 5 | 5 |
| nOC | 15 | 15 | 15 |
| nNO | 0 | 0 | 0 |
| nOO | 6 | 6 | 6 |
| nNC/(nNC + nOC) | 0.25 | 0.25 | 0.25 |
| nNC/(nNC + nOO) | 0.45 | 0.45 | 0.45 |
| nNO/(nNO + nOC) | 0.00 | 0.00 | 0.00 |
| nNO/(nNO + nOO) | 0.00 | 0.00 | 0.00 |
| Number of | 0 | 0 | 0 |
| PEG Monomers | | | |
| Number of | 8 | 8 | 8 |
| PPG Monomers | | | |
| Fatty Acid | 5 | 5 | 5 |
| CH₃(CH₂)ₙCOOH, | | | |
| n = ? | | | |
| Molecular | 651.8 | 665.8 | 693.8 |
| Weight, MwP | | | |
| (g/mol, Dalton) | | | |
| nNC | 7 | 7 | 7 |
| nOC | 24 | 24 | 24 |
| nNO | 0 | 0 | 0 |
| nOO | 8 | 8 | 8 |
| nNC/(nNC + nOC) | 0.23 | 0.23 | 0.23 |
| nNC/(nNC + nOO) | 0.47 | 0.47 | 0.47 |
| nNO/(nNO + nOC) | 0.00 | 0.00 | 0.00 |
| nNO/(nNO + nOO) | 0.00 | 0.00 | 0.00 |
| Number of | 5 | 5 | 5 |
| PEG Monomers | | | |
| Number of | 4 | 4 | 4 |
| PPG Monomers | | | |
| Fatty Acid | 7 | 7 | 7 |
| CH₃(CH₂)ₙCOOH, | | | |
| n = ? | | | |
| Molecular | 667.7 | 681.7 | 709.8 |
| Weight, MwP | | | |
| (g/mol, Dalton) | | | |
| nNC | 9 | 9 | 9 |
| nOC | 22 | 22 | 22 |
| nNO | 0 | 0 | 0 |
| nOO | 9 | 9 | 9 |
| nNC/(nNC + nOC) | 0.29 | 0.29 | 0.29 |
| nNC/(nNC + nOO) | 0.50 | 0.50 | 0.50 |
| nNO/(nNO + nOC) | 0.00 | 0.00 | 0.00 |
| nNO/(nNO + nOO) | 0.00 | 0.00 | 0.00 |
| Number of | 2 | 2 | 2 |
| PEG Monomers | | | |
| Number of | 2 | 2 | 2 |
| PPG Monomers | | | |
| Fatty Acid | 9 | 9 | 9 |
| CH₃(CH₂)ₙCOOH, | | | |
| n = ? | | | |
| Molecular | 447.4 | 461.4 | 489.5 |
| Weight, MwP | | | |
| (g/mol, Dalton) | | | |
| nNC | 11 | 11 | 11 |
| nOC | 10 | 10 | 10 |
| nNO | 0 | 0 | 0 |
| nOO | 4 | 4 | 4 |
| nNC/(nNC + nOC) | 0.52 | 0.52 | 0.52 |
| nNC/(nNC + nOO) | 0.73 | 0.73 | 0.73 |
| nNO/(nNO + nOC) | 0.00 | 0.00 | 0.00 |
| nNO/(nNO + nOO) | 0.00 | 0.00 | 0.00 |
| Number of | 5 | 5 | 5 |
| PEG Monomers | | | |
| Number of | 2 | 2 | 2 |
| PPG Monomers | | | |
| Fatty Acid | 11 | 11 | 11 |
| CH₃(CH₂)ₙCOOH, | | | |
| n = ? | | | |
| Molecular | 607.6 | 621.6 | 649.6 |
| Weight, MwP | | | |
| (g/mol, Dalton) | | | |
| nNC | 13 | 13 | 13 |
| nOC | 16 | 16 | 16 |
| nNO | 0 | 0 | 0 |
| nOO | 7 | 7 | 7 |
| nNC/(nNC + nOC) | 0.45 | 0.45 | 0.45 |
| nNC/(nNC + nOO) | 0.65 | 0.65 | 0.65 |
| nNO/(nNO + nOC) | 0.00 | 0.00 | 0.00 |
| nNO/(nNO + nOO) | 0.00 | 0.00 | 0.00 |
| Number of | 0 | 0 | 0 |
| PEG Monomers | | | |
| Number of | 5 | 5 | 5 |
| PPG Monomers | | | |
| Fatty Acid | 13 | 13 | 13 |
| CH₃(CH₂)ₙCOOH, | | | |
| n = ? | | | |
| Molecular | 589.5 | 603.5 | 631.6 |
| Weight, MwP | | | |
| (g/mol, Dalton) | | | |
| nNC | 15 | 15 | 15 |
| nOC | 15 | 15 | 15 |
| nNO | 0 | 0 | 0 |
| nOO | 5 | 5 | 5 |
| nNC/(nNC + nOC) | 0.50 | 0.50 | 0.50 |
| nNC/(nNC + nOO) | 0.75 | 0.75 | 0.75 |
| nNO/(nNO + nOC) | 0.00 | 0.00 | 0.00 |
| nNO/(nNO + nOO) | 0.00 | 0.00 | 0.00 |
| Number of | 2 | 2 | 2 |
| PEG Monomers | | | |
| Number of | 2 | 2 | 2 |
| PPG Monomers | | | |
| Fatty Acid | 15 | 15 | 15 |
| CH₃(CH₂)ₙCOOH, | | | |
| n = ? | | | |
| Molecular | 531.4 | 545.4 | 573.5 |
| Weight, MwP | | | |
| (g/mol, Dalton) | | | |
| nNC | 17 | 17 | 17 |
| nOC | 10 | 10 | 10 |
| nNO | 0 | 0 | 0 |
| nOO | 4 | 4 | 4 |
| nNC/(nNC + nOC) | 0.63 | 0.63 | 0.63 |
| nNC/(nNC + nOO) | 0.81 | 0.81 | 0.81 |
| nNO/(nNO + nOC) | 0.00 | 0.00 | 0.00 |
| nNO/(nNO + nOO) | 0.00 | 0.00 | 0.00 |
| Number of | 0 | 0 | 0 |
| PEG Monomers | | | |
| Number of | 0 | 0 | 0 |
| PPG Monomers | | | |
| Fatty Acid | 34 | 34 | 34 |
| CH₃(CH₂)ₙCOOH, | | | |
| n = ? | | | |
| Molecular | 593.1 | 607.2 | 635.2 |
| Weight, MwP | | | |
| (g/mol, Dalton) | | | |
| nNC | 36 | 36 | 36 |
| nOC | 0 | 0 | 0 |
| nNO | 0 | 0 | 0 |
| nOO | 0 | 0 | 0 |
| nNC/(nNC + nOC) | 1.00 | 1.00 | 1.00 |
| nNC/(nNC + nOO) | 1.00 | 1.00 | 1.00 |
| nNO/(nNO + nOC) | — | — | — |
| nNO/(nNO + nOO) | — | — | — |

| | 12th Example | 13th Example | 14th Example |
|---|---|---|---|
| IUPAC Name of | Leucine | Isoleucine | Phenylala-nine |
| Central Structure | | | |
| Structural Formula | 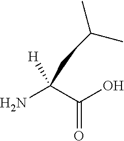 | 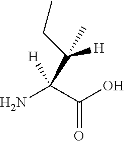 | 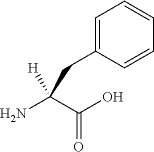 [SMILES: N[C@@H](Cc1ccccc1)C(=O)O] |
| Molecular Weight | 131.17 | 131.17 | 165.19 |
| MwP (g/mol) | | | |
| Abbreviation 1 | Leu | Ile | Phe |
| Abbreviation 2 | L | I | F |
| Residue Property | Hydrophobic | Hydrophobic | Hydrophobic |
| Representative Structure as Side-Chain Structure Being PEG | 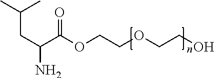 | 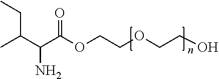 | 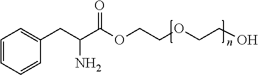 |
| Representative Structure as Side-Chain Structure Being PPG | 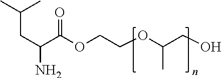 | 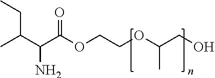 | 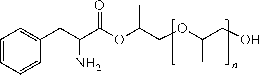 |
| Representative Structure as Side-Chain Structure Being Fatty Acid (CH₃(CH₂)ₙCOOH, n ≥ 3) | 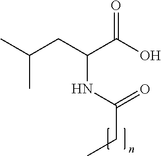 | 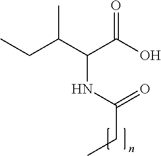 | 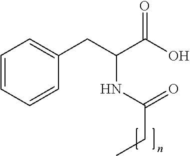 |

Side-Chain Structure Being PEG and PPG

| | | | |
|---|---|---|---|
| Number of | 12 | 12 | 12 |
| PEG Monomers | | | |
| Number of | 0 | 0 | 0 |
| PPG Monomers | | | |
| Molecular | 659.8 | 659.8 | 693.8 |
| Weight, MwP | | | |
| (g/mol, Dalton) | | | |
| nNC | 0 | 0 | 0 |
| nOC | 24 | 24 | 24 |
| nNO | 0 | 0 | 0 |
| nOO | 12 | 12 | 12 |
| nNC/(nNC + nOC) | 0 | 0 | 0 |
| nOC/nOO | 2 | 2 | 2 |
| nNO/(nNO + nOC) | 0 | 0 | 0 |
| nNO/(nNO + nOO) | 0 | 0 | 0 |
| Number of | 0 | 0 | 0 |
| PEG Monomers | | | |
| Number of | 12 | 12 | 12 |
| PPG Monomers | | | |
| Molecular | 828.1 | 828.1 | 862.1 |
| Weight, MwP | | | |
| (g/mol, Dalton) | | | |
| nNC | 0 | 0 | 0 |
| nOC | 36 | 36 | 36 |
| nNO | 0 | 0 | 0 |
| nOO | 12 | 12 | 12 |
| nNC/(nNC + nOC) | 0 | 0 | 0 |
| nOC/nOO | 3 | 3 | 3 |
| nNO/(nNO + nOC) | 0 | 0 | 0 |
| nNO/(nNO + nOO) | 0 | 0 | 0 |
| Number of | 6 | 6 | 6 |
| PEG Monomers | | | |
| Number of | 6 | 6 | 6 |
| PPG Monomers | | | |
| Molecular | 744.0 | 744.0 | 778.0 |
| Weight, MwP | | | |
| (g/mol, Dalton) | | | |
| nNC | 0 | 0 | 0 |
| nOC | 30 | 30 | 30 |
| nNO | 0 | 0 | 0 |
| nOO | 12 | 12 | 12 |
| nNC/(nNC + nOC) | 0 | 0 | 0 |
| nOC/nOO | 3 | 3 | 3 |
| nNO/(nNO + nOC) | 0 | 0 | 0 |
| nNO/(nNO + nOO) | 0 | 0 | 0 |
| Number of | 2 | 2 | 2 |
| PEG Monomers | | | |
| Number of | 5 | 5 | 5 |
| PPG Monomers | | | |
| Molecular | 509.7 | 509.7 | 543.7 |
| Weight, MwP | | | |
| (g/mol, Dalton) | | | |
| nNC | 0 | 0 | 0 |
| nOC | 19 | 19 | 19 |
| nNO | 0 | 0 | 0 |
| nOO | 7 | 7 | 7 |
| nNC/(nNC + nOC) | 0 | 0 | 0 |
| nOC/nOO | 3 | 3 | 3 |
| nNO/nNO + nOC) | 0 | 0 | 0 |
| nNO/nNO + nOO) | 0 | 0 | 0 |
| Number of | 5 | 5 | 5 |
| PEG Monomers | | | |
| Number of | 2 | 2 | 2 |
| PPG Monomers | | | |
| Molecular | 467.6 | 467.6 | 501.6 |
| Weight, MwP | | | |
| (g/mol, Dalton) | | | |
| nNC | 0 | 0 | 0 |
| nOC | 16 | 16 | 16 |
| nNO | 0 | 0 | 0 |
| nOO | 7 | 7 | 7 |
| nNC/nNC + nOC) | 0 | 0 | 0 |
| nOC/nOO | 2 | 2 | 2 |
| nNO/(nNO + nOC) | 0 | 0 | 0 |
| nNO/(nNO + nOO) | 0 | 0 | 0 |
| Number of | 5 | 5 | 5 |
| PEG Monomers | | | |
| Number of | 5 | 5 | 5 |
| PPG Monomers | | | |
| Molecular | 641.8 | 641.8 | 675.9 |
| Weight, MwP | | | |
| (g/mol, Dalton) | | | |
| nNc | 0 | 0 | 0 |
| nOC | 25 | 25 | 25 |
| nNO | 0 | 0 | 0 |
| nOO | 10 | 10 | 10 |
| nNC/(nNC + nOC) | 0 | 0 | 0 |
| nOC/nOO | 3 | 3 | 3 |
| nNO/(nNO + nOC) | 0 | 0 | 0 |
| nNO/(nNO + nOO) | 0 | 0 | 0 |
| Number of | 4 | 4 | 4 |
| PEG Monomers | | | |
| Number of | 4 | 4 | 4 |
| PPG Monomers | | | |
| Molecular | 539.7 | 539.7 | 573.7 |
| Weight, MwP | | | |
| (g/mol, Dalton) | | | |
| nNC | 0 | 0 | 0 |
| nOC | 20 | 20 | 20 |
| nNO | 0 | 0 | 0 |
| nOO | 8 | 8 | 8 |
| nNC/(nNC + nOC) | 0 | 0 | 0 |
| nOC/nOO | 3 | 3 | 3 |
| nNO/(nNO + nOC) | 0 | 0 | 0 |
| nNO/(nNO + nOO) | 0 | 0 | 0 |
| Number of | 8 | 8 | 8 |
| PEG Monomers | | | |
| Number of | 8 | 8 | 8 |
| PPG Monomers | | | |
| Molecular | 948.2 | 948.2 | 982.2 |
| Weight, MwP | | | |
| (g/mol, Dalton) | | | |
| nNC | 0 | 0 | 0 |
| nOC | 40 | 40 | 40 |
| nNO | 0 | 0 | 0 |
| nOO | 16 | 16 | 16 |
| nNC/(nNC + nOC) | 0 | 0 | 0 |
| nOC/nOO | 3 | 3 | 3 |
| nNO/(nNO + nOC) | 0 | 0 | 0 |
| nNO/(nNO + nOO) | 0 | 0 | 0 |

Side-Chain Structure Being PEG, PPG and Fatty Acid

| | | | |
|---|---|---|---|
| Number of | 3 | 3 | 3 |
| PEG Monomers | | | |
| Number of | 3 | 3 | 3 |
| PPG Monomers | | | |
| Fatty Acid | 3 | 3 | 3 |
| CH₃(CH₂)ₙCOOH, | | | |
| n = ? | | | |
| Molecular | 521.6 | 521.6 | 555.6 |
| Weight, MwP | | | |
| (g/mol, Dalton) | | | |
| nNC | 5 | 6 | 5 |
| nOC | 15 | 15 | 15 |
| nNO | 0 | 0 | 0 |
| nOO | 6 | 6 | 6 |
| nNC/(nNC + nOC) | 0.25 | 0.25 | 0.25 |
| nNC/(nNC + nOO) | 0.45 | 0.45 | 0.45 |
| nNO/(nNO + nOC) | 0.00 | 0.00 | 0.00 |
| nNO/(nNO + nOO) | 0.00 | 0.00 | 0.00 |
| Number of | 0 | 0 | 0 |
| PEG Monomers | | | |
| Number of | 8 | 8 | 8 |
| PPG Monomers | | | |
| Fatty Acid | 5 | 5 | 5 |
| CH₃(CH₂)ₙCOOH, | | | |
| n = ? | | | |
| Molecular | 707.9 | 707.9 | 741.9 |
| Weight, MwP | | | |
| (g/mol, Dalton) | | | |
| nNC | 7 | 7 | 7 |
| nOC | 24 | 24 | 24 |
| nNO | 0 | 0 | 0 |
| nOO | 8 | 8 | 8 |
| nNC/nNC + nOC) | 0.23 | 0.23 | 0.23 |
| nNC/nNC + nOO) | 0.47 | 0.47 | 0.47 |
| nNO/(nNO + nOC) | 0.00 | 0.00 | 0.00 |
| nNO/(nNO + nOO) | 0.00 | 0.00 | 0.00 |
| Number of | 5 | 5 | 5 |
| PEG Monomers | | | |
| Number of | 4 | 4 | 4 |
| PPG Monomers | | | |
| Fatty Acid | 7 | 7 | 7 |
| CH₃(CH₂)ₙCOOH, | | | |
| n = ? | | | |
| Molecular | 723.8 | 723.8 | 757.8 |
| Weight, MwP | | | |
| (g/mol, Dalton) | | | |
| nNC | 9 | 9 | 9 |
| nOC | 22 | 22 | 22 |
| nNO | 0 | 0 | 0 |
| nOO | 9 | 9 | 9 |
| nNC/(nNC + nOC) | 0.29 | 0.29 | 0.29 |
| nNC/(nNC + nOO) | 0.50 | 0.50 | 0.50 |
| nNO/(nNO + nOC) | 0.00 | 0.00 | 0.00 |
| nNO/(nNO + nOO) | 0.00 | 0.00 | 0.00 |
| Number of | 2 | 2 | 2 |
| PEG Monomers | | | |
| Number of | 2 | 2 | 2 |
| PPG Monomers | | | |
| Fatty Acid | 9 | 9 | 9 |
| CH₃(CH₂), COOH, | | | |
| n = ? | | | |
| Molecular | 503.5 | 503.5 | 537.5 |
| Weight, MwP | | | |
| (g/mol, Dalton) | | | |
| nNC | 11 | 11 | 11 |
| nOC | 10 | 10 | 10 |
| nNO | 0 | 0 | 0 |
| nOO | 4 | 4 | 4 |
| nNC/(nNC + nOC) | 0.52 | 0.52 | 0.52 |
| nNC/(nNC + nOO) | 0.73 | 0.73 | 0.73 |
| nNO/(nNO + nOC) | 0.00 | 0.00 | 0.00 |
| nNO/(nNO + nOO) | 0.00 | 0.00 | 0.00 |
| Number of | 5 | 5 | 5 |
| PEG Monomers | | | |
| Number of | 2 | 2 | 2 |
| PPG Monomers | | | |
| Fatty Acid | 11 | 11 | 11 |
| CH₃(CH₂)ₙCOOH, | | | |
| n = ? | | | |
| Molecular | 663.7 | 663.7 | 697.7 |
| Weight, MwP | | | |
| (g/mol, Dalton) | | | |
| nNC | 13 | 13 | 13 |
| nOC | 16 | 16 | 16 |
| nNO | 0 | 0 | 0 |
| nOO | 7 | 7 | 7 |
| nNC/(nNC + nOC) | 0.45 | 0.45 | 0.45 |
| nNC/(nNC + nOO) | 0.65 | 0.65 | 0.65 |
| nNO/(nNO + nOC) | 0.00 | 0.00 | 0.00 |
| nNO/(nNO + nOO) | 0.00 | 0.00 | 0.00 |
| Number of | 0 | 0 | 0 |
| PEG Monomers | | | |
| Number of | 5 | 5 | 5 |
| PPG Monomers | | | |
| Fatty Acid | 13 | 13 | 13 |
| CH₃(CH₂)ₙCOOH, | | | |
| n = ? | | | |
| Molecular | 645.6 | 645.6 | 679.6 |
| Weight, MwP | | | |
| (g/mol, Dalton) | | | |
| nNC | 15 | 15 | 15 |
| nOC | 15 | 15 | 15 |
| nNO | 0 | 0 | 0 |
| nOO | 5 | 5 | 5 |
| nNC/(nNC + nOC) | 0.50 | 0.50 | 0.50 |
| nNC/(nNC + nOO) | 0.75 | 0.75 | 0.75 |
| nNO/(nNO + nOC) | 0.00 | 0.00 | 0.00 |
| nNO/(nNO + nOO) | 0.00 | 0.00 | 0.00 |
| Number of | 2 | 2 | 2 |
| PEG Monomers | | | |
| Number of | 2 | 2 | 2 |
| PPG Monomers | | | |
| Fatty Acid | 15 | 15 | 15 |
| CH₃(CH₂)ₙCOOH, | | | |
| n = ? | | | |
| Molecular | 587.5 | 587.5 | 621.5 |
| Weight, MwP | | | |
| (g/mol, Dalton) | | | |
| nNC | 17 | 17 | 17 |
| nOC | 10 | 10 | 10 |
| nNO | 0 | 0 | 0 |
| nOO | 4 | 4 | 4 |
| nNC/(nNC + nOC) | 0.63 | 0.63 | 0.63 |
| nNC/(nNC + nOO) | 0.81 | 0.81 | 0.81 |
| nNO/(nNO + nOC) | 0.00 | 0.00 | 0.00 |
| nNO/(nNO + nOO) | 0.00 | 0.00 | 0.00 |
| Number of | 0 | 0 | 0 |
| PEG Monomers | | | |
| Number of | 0 | 0 | 0 |
| PPG Monomers | | | |
| Fatty Acid | 34 | 34 | 34 |
| CH₃(CH₂)ₙCOOH, | | | |
| n = ? | | | |
| Molecular | 649.2 | 649.2 | 683.3 |
| Weight, MwP | | | |
| (g/mol, Dalton) | | | |
| nNC | 36 | 36 | 36 |
| nOC | 0 | 0 | 0 |
| nNO | 0 | 0 | 0 |
| nOO | 0 | 0 | 0 |
| nNC/(nNC + nOC) | 1.00 | 1.00 | 1.00 |
| nNC/(nNC + nOO) | 1.00 | 1.00 | 1.00 |
| nNO/(nNO + nOC) | — | — | — |
| nNO/(nNO + nOO) | — | — | — |

| | 15th Example | 16th Example |
|---|---|---|
| IUPAC Name of | Tryptophan | Tyrosine |
| Central Structure | | |
| Structural Formula | 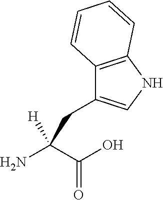 [SMILES: N[C@@H](Cc1c[nH]c2ccccc12)C(=O)O] | 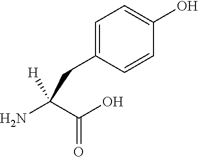 [SMILES: N[C@@H](Cc1ccc(O)cc1)C(=O)O] |
| Molecular Weight | 204.23 | 181.19 |
| MwP (g/mol) | | |
| Abbreviation 1 | Trp | Tyr |
| Abbreviation 2 | W | Y |
| Residue Property | Hydrophobic | Hydrophilic |
| Representative Structure as Side-Chain Structure Being PEG | 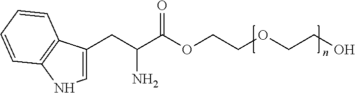 | 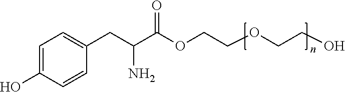 |
| Representative Structure as Side-Chain Structure Being PPG | 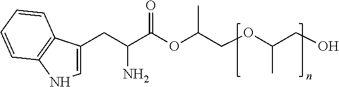 | 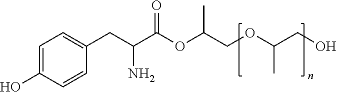 |
| Representative Structure 1 as Side-Chain Structure Being Fatty Acid (CH₃(CH₂)ₙCOOH, n ≥ 3) | 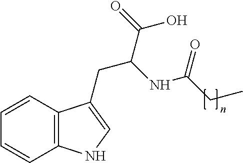 | 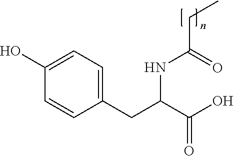 |
| Representative Structure 2 as Side-Chain Structure Being Fatty Acid (CH₃(CH₂)ₙCOOH, n ≥ 3) | — | 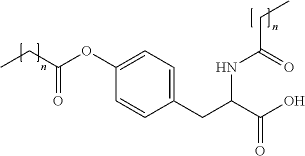 |
| Representative Structure 3 as Side-Chain Structure Being Fatty Acid (CH₃(CH₂)ₙCOOH, n ≥ 3) | — | 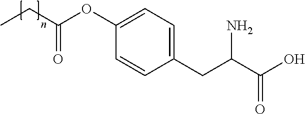 |

Side-Chain Structure Being PEG and PPG

| | | |
|---|---|---|
| Number of | 12 | 12 |
| PEG Monomers | | |
| Number of | 0 | 0 |
| PPG Monomers | | |
| Molecular | 732.9 | 709.8 |
| Weight, MwP | | |
| (g/mol, Dalton) | | |
| nNC | 0 | 0 |
| nOC | 24 | 24 |
| nNO | 0 | 0 |
| nOO | 12 | 12 |
| nNC/(nNC + nOC) | 0 | 0 |
| nOC/nOO | 2 | 2 |
| nNO/(nNO + nOC) | 0 | 0 |
| nNO/(nNO + nOO) | 0 | 0 |
| Number of | 0 | 0 |
| PEG Monomers | | |
| Number of | 12 | 12 |
| PPG Monomers | | |
| Molecular | 901.2 | 878.1 |
| Weight, MwP | | |
| (g/mol, Dalton) | | |
| nNC | 0 | 0 |
| nOC | 36 | 36 |
| nNO | 0 | 0 |
| nOO | 12 | 12 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 6 | 6 |
| PEG Monomers | | |
| Number of | 6 | 6 |
| PPG Monomers | | |
| Molecular | 817.0 | 794.0 |
| Weight, MwP | | |
| (g/mol, Dalton) | | |
| nNC | 0 | 0 |
| nOC | 30 | 30 |
| nNO | 0 | 0 |
| nOO | 12 | 12 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 2 | 2 |
| PEG Monomers | | |
| Number of | 5 | 5 |
| PPG Monomers | | |
| Molecular | 582.7 | 559.7 |
| Weight, MwP | | |
| (g/mol, Dalton) | | |
| nNC | 0 | 0 |
| nOC | 19 | 19 |
| nNO | 0 | 0 |
| nOO | 7 | 7 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 5 | 5 |
| PEG Monomers | | |
| Number of | 2 | 2 |
| PPG Monomers | | |
| Molecular | 540.7 | 517.6 |
| Weight, MwP | | |
| (g/mol, Dalton) | | |
| nNC | 0 | 0 |
| nOC | 16 | 16 |
| nNO | 0 | 0 |
| nOO | 7 | 7 |
| nNC/(nNC + nOC) | 0 | 0 |
| nOC/nOO | 2 | 2 |
| nNO/(nNO + nOC) | 0 | 0 |
| nNO/(nNO + nOO) | 0 | 0 |
| Number of | 5 | 5 |
| PEG Monomers | | |
| Number of | 5 | 5 |
| PPG Monomers | | |
| Molecular | 714.9 | 691.9 |
| Weight, MwP | | |
| (g/mol, Dalton) | | |
| nNC | 0 | 0 |
| nOC | 25 | 25 |
| nNO | 0 | 0 |
| nOO | 10 | 10 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 4 | 4 |
| PEG Monomers | | |
| Number of | 4 | 4 |
| PPG Monomers | | |
| Molecular | 612.8 | 589.7 |
| Weight, MwP | | |
| (g/mol, Dalton) | | |
| nNC | 0 | 0 |
| nOC | 20 | 20 |
| nNO | 0 | 0 |
| nOO | 8 | 8 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 8 | 8 |
| PEG Monomers | | |
| Number of | 8 | 8 |
| PPG Monomers | | |
| Molecular | 1021.3 | 998.2 |
| Weight, MwP | | |
| (g/mol, Dalton) | | |
| nNC | 0 | 0 |
| nOC | 40 | 40 |
| nNO | 0 | 0 |
| nOO | 16 | 16 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |

Side-Chain Structure Being PEG, PPG and Fatty Acid

| | | |
|---|---|---|
| Number of | 3 | 3 |
| PEG Monomers | | |
| Number of | 3 | 3 |
| PPG Monomers | | |
| Fatty Acid | 3 | 3 |
| CH₃(CH₂)ₙCOOH, | | |
| n = ? | | |
| Molecular | 594.7 | 571.6 |
| Weight, MwP | | |
| (g/mol, Dalton) | | |
| nNC | 5 | 5 |
| nOC | 15 | 15 |
| nNO | 0 | 0 |
| nOO | 6 | 6 |
| nNC/(nNC + nOC) | 0.25 | 0.25 |
| nNC/(nNC + nOO) | 0.45 | 0.45 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 8 | 8 |
| PPG Monomers | | |
| Fatty Acid | 5 | 5 |
| CH₃(CH₂)ₙCOOH, | | |
| n = ? | | |
| Molecular | 780.9 | 757.9 |
| Weight, MwP | | |
| (g/mol, Dalton) | | |
| nNC | 7 | 7 |
| nOC | 24 | 24 |
| nNO | 0 | 0 |
| nOO | 8 | 8 |
| nNC/(nNC + nOC) | 0.23 | 0.23 |
| nNC/(nNC + nOO) | 0.47 | 0.47 |
| nNO/(nNO + nOC) | 0.00 | 0.00 |
| nNO/(nNO + nOO) | 0.00 | 0.00 |
| Number of | 5 | 6 |
| PEG Monomers | | |
| Number of | 4 | 4 |
| PPG Monomers | | |
| Fatty Acid | 7 | 7 |
| CH₃(CH₂)ₙCOOH, | | |
| n = ? | | |
| Molecular | 796.9 | 773.8 |
| Weight, MwP | | |
| (g/mol, Dalton) | | |
| nNC | 9 | 9 |
| nOC | 22 | 22 |
| nNO | 0 | 0 |
| nOO | 9 | 9 |
| nNC/n(NC + nOC) | 0.29 | 0.29 |
| nNC/(nNC + nOO) | 0.50 | 0.50 |
| nNO/(nNO + nOC) | 0.00 | 0.00 |
| nNO/(nNO + nOO) | 0.00 | 0.00 |
| Number of | 2 | 2 |
| PEG Monomers | | |
| Number of | 2 | 2 |
| PPG Monomers | | |
| Fatty Acid | 9 | 9 |
| CH₃(CH₂)ₙCOOH, | | |
| n = ? | | |
| Molecular | 576.6 | 553.5 |
| Weight, MwP | | |
| (g/mol, Dalton) | | |
| nNC | 11 | 11 |
| nOC | 10 | 10 |
| nNO | 0 | 0 |
| nOO | 4 | 4 |
| nNC/(nNC + nOC) | 0.62 | 0.52 |
| nNC/(nNC + nOO) | 0.73 | 0.73 |
| nNO/(nNO + nOC) | 0.00 | 0.00 |
| nNO/(nNO + nOO) | 0.00 | 0.00 |
| Number of | 6 | 5 |
| PEG Monomers | | |
| Number of | 2 | 2 |
| PPG Monomers | | |
| Fatty Acid | 11 | 11 |
| CH₃(CH₂)ₙCOOH | | |
| n = ? | | |
| Molecular | 736.7 | 713.7 |
| Weight, MwP | | |
| (g/mol, Dalton) | | |
| nNC | 13 | 13 |
| nOC | 16 | 16 |
| nNO | 0 | 0 |
| nOO | 7 | 7 |
| nNC/(nNC + nOC) | 0.45 | 0.45 |
| nNC/(nNC + nOO) | 0.65 | 0.65 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 5 | 5 |
| PPG Monomers | | |
| Fatty Acid | 13 | 13 |
| CH₃(CH₂)ₙCOOH, | | |
| n = ? | | |
| Molecular | 718.7 | 695.6 |
| Weight, MwP | | |
| (g/mol, Dalton) | | |
| nNC | 15 | 15 |
| nOC | 15 | 15 |
| nNO | 0 | 0 |
| nOO | 5 | 5 |
| nNC/(nNC + nOC) | 0.50 | 0.50 |
| nNC/(nNC + nOO) | 0.75 | 0.75 |
| nNO/(nNO + nOC) | 0.00 | 0.00 |
| nNO/(nNO + nOO) | 0.00 | 0.00 |
| Number of | 2 | 2 |
| PEG Monomers | | |
| Number of | 2 | 2 |
| PPG Monomers | | |
| Fatty Acid | 15 | 15 |
| CH₃(CH₂)ₙCOOH, | | |
| n = ? | | |
| Molecular | 660.6 | 637.5 |
| Weight, MwP | | |
| (g/mol, Dalton) | | |
| nNC | 17 | 17 |
| nOC | 10 | 10 |
| nNO | 0 | 0 |
| nOO | 4 | 4 |
| nNC/(nNC + nOC) | 0.63 | 0.63 |
| nNC/(nNC + nOO) | 0.81 | 0.81 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 0 | 0 |
| PPG Monomers | | |
| Fatty Acid | 34 | 34 |
| CH₃(CH₂)ₙCOOH, | | |
| n = ? | | |
| Molecular | 722.3 | 699.3 |
| Weight, MwP | | |
| (g/mol, Dalton) | | |
| nNC | 36 | 36 |
| nOC | 0 | 0 |
| nNO | 0 | 0 |
| nOO | 0 | 0 |
| nNC/(nNC + nOC) | 1.00 | 1.00 |
| nNC/(nNC + nOO) | 1.00 | 1.00 |
| nNO/(nNO + nOC) | — | — |
| nNO/(nNO + nOO) | — | — |

| | 17th Example | 18th Example |
|---|---|---|
| IUPAC Name of | Aspartate | Histidine |
| Central Structure | | |
| Structural Formula | 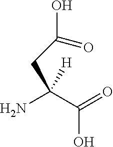 | 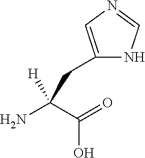 [SMILES: N[C@@H](Cc1cnc[nH]1)C(=O)O] |
| Molecular Weight | 133.1 | 155.16 |
| MwP (g/mol) | | |
| Abbreviation 1 | Asp | His |
| Abbreviation 2 | D | H |
| Residue Property | Acidic | Basic |
| Representative Structure 1 as Side-Chain Structure Being PEG | 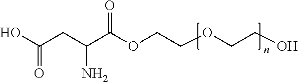 | 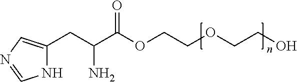 |
| Representative Structure 2 as Side-Chain Structure Being PEG | 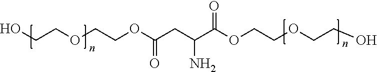 | — |
| Representative Structure 1 as Side-Chain Structure Being PPG | 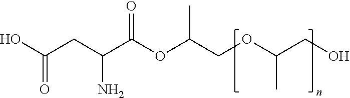 | 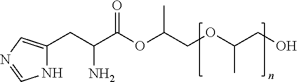 |
| Representative Structure 2 as Side-Chain Structure Being PPG | 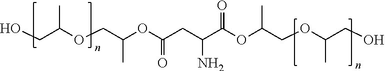 | — |
| Representative Structure as Side-Chain Structure Being Fatty Acid (CH₃(CH₂)ₙCOOH, H, n ≥ 3) | 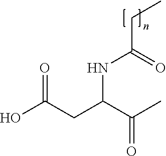 | 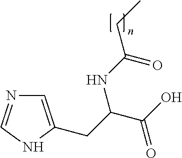 |

Side-Chain Structure Being PEG and PPG

| | | |
|---|---|---|
| Number of | 12 | 12 |
| PEG Monomers | | |
| Number of | 0 | 0 |
| PPG Monomers | | |
| Molecular | 661.7 | 683.8 |
| Weight, MwP | | |
| (g/mol, Dalton) | | |
| nNC | 0 | 0 |
| nOC | 24 | 24 |
| nNO | 0 | 0 |
| nOO | 12 | 12 |
| nNC/(nNC + nOC) | 0 | 0 |
| nOC/nOO | 2 | 2 |
| nNO/(nNO + nOC) | 0 | 0 |
| nNO/(nNO + nOO) | 0 | 0 |
| Number of | 0 | 0 |
| PEG Monomers | | |
| Number of | 12 | 12 |
| PPG Monomers | | |
| Molecular | 830.0 | 852.1 |
| Weight, MwP | | |
| (g/mol, Dalton) | | |
| nNC | 0 | 0 |
| nOC | 36 | 36 |
| nNO | 0 | 0 |
| nOO | 12 | 12 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 6 | 6 |
| PEG Monomers | | |
| Number of | 5 | 5 |
| PPG Monomers | | |
| Molecular | 745.9 | 768.0 |
| Weight, MwP | | |
| (g/mol, Dalton) | | |
| nNC | 0 | 0 |
| nOC | 30 | 30 |
| nNO | 0 | 0 |
| nOO | 12 | 12 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 2 | 2 |
| PEG Monomers | | |
| Number of | 6 | 6 |
| PPG Monomers | | |
| Molecular | 511.6 | 533.7 |
| Weight, MwP | | |
| (g/mol, Dalton) | | |
| nNC | 0 | 0 |
| nOC | 19 | 19 |
| nNO | 0 | 0 |
| nOO | 7 | 7 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 5 | 5 |
| PEG Monomers | | |
| Number of | 2 | 2 |
| PPG Monomers | | |
| Molecular | 469.5 | 491.6 |
| Weight, MwP | | |
| (g/mol, Dalton) | | |
| nNC | 0 | 0 |
| nOC | 16 | 16 |
| nNO | 0 | 0 |
| nOO | 7 | 7 |
| nNC/(nNC + nOC) | 0 | 0 |
| nOC/nOO | 2 | 2 |
| nNO/(nNO + nOC) | 0 | 0 |
| nNO/(nNO + nOO) | 0 | 0 |
| Number of | 5 | 5 |
| PEG Monomers | | |
| Number of | 5 | 5 |
| PPG Monomers | | |
| Molecular | 643.8 | 665.8 |
| Weight, MwP | | |
| (g/mol, Dalton) | | |
| nNC | 0 | 0 |
| nOC | 25 | 25 |
| nNO | 0 | 0 |
| nOO | 10 | 10 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 4 | 4 |
| PEG Monomers | | |
| Number of | 4 | 4 |
| PPG Monomers | | |
| Molecular | 541.6 | 563.7 |
| Weight, MwP | | |
| (g/mol, Dalton) | | |
| nNC | 0 | 0 |
| nOC | 20 | 20 |
| nNO | 0 | 0 |
| nOO | 8 | 8 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 8 | 8 |
| PEG Monomers | | |
| Number of | 8 | 8 |
| PPG Monomers | | |
| Molecular | 950.2 | 972.2 |
| Weight, MwP | | |
| (g/mol, Dalton) | | |
| nNC | 0 | 0 |
| nOC | 40 | 40 |
| nNO | 0 | 0 |
| nOO | 16 | 16 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |

Side-Chain Structure Being PEG, PPG and Fatty Acid

| | | |
|---|---|---|
| Number of | 3 | 3 |
| PEG Monomers | | |
| Number of | 3 | 3 |
| PPG Monomers | | |
| Fatty Acid | 3 | 3 |
| CH₃(CH₂)ₙCOOH, | | |
| n = ? | | |
| Molecular | 523.6 | 545.6 |
| Weight, MwP | | |
| (g/mol, Dalton) | | |
| nNC | 5 | 5 |
| nOC | 15 | 15 |
| nNO | 0 | 0 |
| nOO | 6 | 6 |
| nNC/(nNC + nOC) | 0.25 | 0.25 |
| nNC/(nNC + nOO) | 0.45 | 0.45 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 8 | 8 |
| PPG Monomers | | |
| Fatty Acid | 5 | 5 |
| CH₃(CH₂)ₙCOOH | | |
| n = ? | | |
| Molecular | 709.8 | 731.9 |
| Weight, MwP | | |
| (g/mol, Dalton) | | |
| nNC | 7 | 7 |
| nOC | 24 | 24 |
| nNO | 0 | 0 |
| nOO | 8 | 8 |
| nNC/(nNC + nOC) | 0.23 | 0.23 |
| nNC/(nNC + nOO) | 0.47 | 0.47 |
| nNO/(nNO + nOC) | 0.00 | 0.00 |
| nNO/(nNO + nOO) | 0.00 | 0.00 |
| Number of | 5 | 5 |
| PEG Monomers | | |
| Number of | 4 | 4 |
| PPG Monomers | | |
| Fatty Acid | 7 | 7 |
| CH₃(CH₂)ₙCOOH, | | |
| n = ? | | |
| Molecular | 725.7 | 747.8 |
| Weight, MwP | | |
| (g/mol, Dalton) | | |
| nNC | 9 | 9 |
| nOC | 22 | 22 |
| nNO | 0 | 0 |
| nOO | 9 | 9 |
| nNC/(nNC + nOC) | 0.29 | 0.29 |
| nNC/(nNC + nOO) | 0.50 | 0.50 |
| nNO/(nNO + nOC) | 0.00 | 0.00 |
| nNO/(nNO + nOO) | 0.00 | 0.00 |
| Number of | 2 | 2 |
| PEG Monomers | | |
| Number of | 2 | 2 |
| PPG Monomers | | |
| Fatty Acid | 9 | 9 |
| CH₃(CH₂)ₙCOOH, | | |
| n = ? | | |
| Molecular | 505.4 | 527.5 |
| Weight, MwP | | |
| (g/mol, Dalton) | | |
| nNC | 11 | 11 |
| nOC | 10 | 10 |
| nNO | 0 | 0 |
| nOO | 4 | 4 |
| nNC/(nNC + nOC) | 0.52 | 0.52 |
| nNC/(nNC + nOO) | 0.73 | 0.73 |
| nNO/(nNO + nOC) | 0.00 | 0.00 |
| nNO/(nNO + nOO) | 0.00 | 0.00 |
| Number of | 5 | 5 |
| PEG Monomers | | |
| Number of | 2 | 2 |
| PPG Monomers | | |
| Fatty Acid | 11 | 11 |
| CH₃(CH₂)ₙCOOH, | | |
| n = ? | | |
| Molecular | 665.6 | 687.6 |
| Weight, MwP | | |
| (g/mol, Dalton) | | |
| nNC | 13 | 13 |
| nOC | 16 | 16 |
| nNO | 0 | 0 |
| nOO | 7 | 7 |
| nNC/(nNC + nOC) | 0.45 | 0.45 |
| nNC/(nNC + nOO) | 0.65 | 0.65 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 5 | 5 |
| PPG Monomers | | |
| Fatty Acid | 13 | 13 |
| CH₃(CH₂)ₙCOOH, | | |
| n = ? | | |
| Molecular | 647.6 | 669.6 |
| Weight, MwP | | |
| (g/mol, Dalton) | | |
| nNC | 15 | 15 |
| nOC | 15 | 15 |
| nNO | 0 | 0 |
| nOO | 5 | 5 |
| nNC/(nNC + nOC) | 0.50 | 0.50 |
| nNC/(nNC + nOO) | 0.75 | 0.75 |
| nNO/(nNO + nOC) | 0.00 | 0.00 |
| nNO/(nNO + nOO) | 0.00 | 0.00 |
| Number of | 2 | 2 |
| PEG Monomers | | |
| Number of | 2 | 2 |
| PPG Monomers | | |
| Fatty Acid | 15 | 15 |
| CH₃(CH₂)ₙCOOH, | | |
| n = ? | | |
| Molecular | 589.4 | 611.5 |
| Weight, MwP | | |
| (g/mol, Dalton) | | |
| nNC | 17 | 17 |
| nOC | 10 | 10 |
| nNO | 0 | 0 |
| nOO | 4 | 4 |
| nNC/(nNC + nOC) | 0.63 | 0.63 |
| nNC/(nNC + nOO) | 0.81 | 0.81 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 0 | 0 |
| PPG Monomers | | |
| Fatty Acid | 34 | 34 |
| CH₃(CH₂)ₙCOOH, | | |
| n = ? | | |
| Molecular | 651.2 | 673.2 |
| Weight, MwP | | |
| (g/mol, Dalton) | | |
| nNC | 36 | 36 |
| nOC | 0 | 0 |
| nNO | 0 | 0 |
| nOO | 0 | 0 |
| nNC/(nNC + nOC) | 1.00 | 1.00 |
| nNC/(nNC + nOO) | 1.00 | 1.00 |
| nNO/(nNO + nOC) | — | — |
| nNO/(nNO + nOO) | — | — |

| | 19th Example | 20th Example |
|---|---|---|
| IUPAC Name of | Asparagine | Glutamate |
| Central Structure | | |
| Structural Formula | 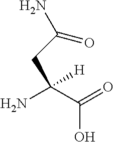 | 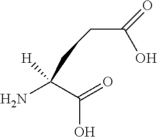 |
| Molecular Weight | 132.12 | 147.13 |
| MwP (g/mol) | | |
| Abbreviation 1 | Asn | Glu |
| Abbreviation 2 | N | E |
| Residue Property | Hydrophilic | Acidic |
| Representative Structure 1 as Side-Chain Structure Being PEG | 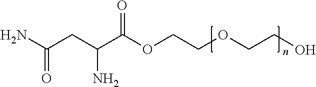 | 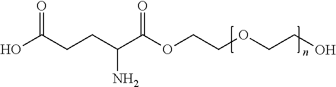 |
| Representative Structure 2 as Side-Chain Structure Being PEG | — | 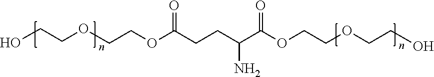 |
| Representative Structure 1 as Side-Chain Structure Being PPG | 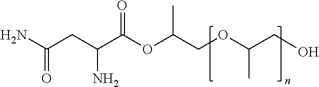 | 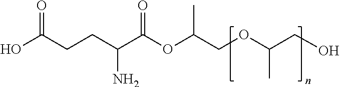 |
| Representative Structure 2 as Side-Chain Structure Being PPG | — | 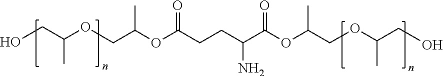 |
| Representative Structure 1 as Side-Chain Structure Being Fatty Acid (CH₃(CH₂)ₙCOOH, n ≥ 3) | 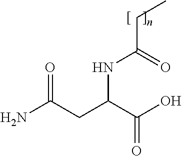 | 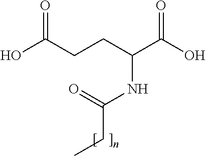 |
| Representative Structure 2 as Side-Chain Structure Being Fatty Acid (CH₃(CH₂)ₙCOOH, | 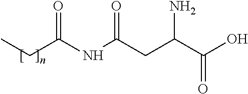 | — |
| n ≥ 3) | | |
| Representative Structure 3 as Side-Chain Structure Being Fatty Acid (CH₃(CH₂)ₙCOOH, n ≥ 3) | 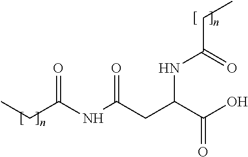 | — |

Side-Chain Structure Being PEG and PPG

| | | |
|---|---|---|
| Number of | 12 | 12 |
| PEG Monomers | | |
| Number of | 0 | 0 |
| PPG Monomers | | |
| Molecular | 660.8 | 675.8 |
| Weight, MwP | | |
| (g/mol, Dalton) | | |
| nNC | 0 | 0 |
| nOC | 24 | 24 |
| nNO | 0 | 0 |
| nOO | 12 | 12 |
| nNC/(nNC + nOC) | 0 | 0 |
| nOC/nOO | 2 | 2 |
| nNO/(nNO + nOC) | 0 | 0 |
| nNO/(nNO + nOO) | 0 | 0 |
| Number of | 0 | 0 |
| PEG Monomers | | |
| Number of | 12 | 12 |
| PPG Monomers | | |
| Molecular | 829.1 | 844.1 |
| Weight, MwP | | |
| (g/mol, Dalton) | | |
| nNC | 0 | 0 |
| nOC | 36 | 36 |
| nNO | 0 | 0 |
| nOO | 12 | 12 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 6 | 6 |
| PEG Monomers | | |
| Number of | 6 | 6 |
| PPG Monomers | | |
| Molecular | 744.9 | 759.9 |
| Weight, MwP | | |
| (g/mol, Dalton) | | |
| nNC | 0 | 0 |
| nOC | 30 | 30 |
| nNO | 0 | 0 |
| nOO | 12 | 12 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 2 | 2 |
| PEG Monomers | | |
| Number of | 5 | 5 |
| PPG Monomers | | |
| Molecular | 510.6 | 525.6 |
| Weight, MwP | | |
| (g/mol, Dalton) | | |
| nNC | 0 | 0 |
| nOC | 19 | 19 |
| nNO | 0 | 0 |
| nOO | 7 | 7 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 5 | 5 |
| PEG Monomers | | |
| Number of | 2 | 2 |
| PPG Monomers | | |
| Molecular | 468.5 | 483.6 |
| Weight, MwP | | |
| (g/mol, Dalton) | | |
| nNC | 0 | 0 |
| nOC | 16 | 16 |
| nNO | 0 | 0 |
| nOO | 7 | 7 |
| nNC/(nNC + nOC) | 0 | 0 |
| nOC/nOO | 2 | 2 |
| nNO/(nNO + nOC) | 0 | 0 |
| nNO/(nNO + nOO) | 0 | 0 |
| Number of | 5 | 5 |
| PEG Monomers | | |
| Number of | 5 | 5 |
| PPG Monomers | | |
| Molecular | 642.8 | 657.8 |
| Weight, MwP | | |
| (g/mol, Dalton) | | |
| nNC | 0 | 0 |
| nOC | 25 | 25 |
| nNO | 0 | 0 |
| nOO | 10 | 10 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 4 | 4 |
| PEG Monomers | | |
| Number of | 4 | 4 |
| PPG Monomers | | |
| Molecular | 540.6 | 555.7 |
| Weight, MwP | | |
| (g/mol, Dalton) | | |
| nNC | 0 | 0 |
| nOC | 20 | 20 |
| nNO | 0 | 0 |
| nOO | 8 | 8 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 8 | 8 |
| PEG Monomers | | |
| Number of | 8 | 8 |
| PPG Monomers | | |
| Molecular | 949.2 | 964.2 |
| Weight, MwP | | |
| (g/mol, Dalton) | | |
| nNC | 0 | 0 |
| nOC | 40 | 40 |
| nNO | 0 | 0 |
| nOO | 16 | 16 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |

Side-Chain Structure Being PEG, PPG and Fatty Acid

| | | |
|---|---|---|
| Number of | 3 | 3 |
| PEG Monomers | | |
| Number of | 3 | 3 |
| PPG Monomers | | |
| Fatty Acid | 3 | 3 |
| CH₃(CH₂)ₙCOOH, | | |
| n = ? | | |
| Molecular | 522.6 | 537.6 |
| Weight, MwP | | |
| (g/mol, Dalton) | | |
| nNC | 5 | 5 |
| nOC | 15 | 15 |
| nNO | 0 | 0 |
| nOO | 6 | 6 |
| nNC/(nNC + nOC) | 0.25 | 0.25 |
| nNC/(nNC + nOO) | 0.45 | 0.45 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 8 | 8 |
| PPG Monomers | | |
| Fatty Acid | 5 | 5 |
| CH₃(CH₂)ₙCOOH, | | |
| n = ? | | |
| Molecular | 708.8 | 723.8 |
| Weight, MwP | | |
| (g/mol, Dalton) | | |
| nNC | 7 | 7 |
| nOC | 24 | 24 |
| nNO | 0 | 0 |
| nOO | 8 | 8 |
| nNC/(nNC + nOC) | 0.23 | 0.23 |
| nNC/(nNC + nOO) | 0.47 | 0.47 |
| nNO/(nNO + nOC) | 0.00 | 0.00 |
| nNO/(nNO + nOO) | 0.00 | 0.00 |
| Number of | 5 | 5 |
| PEG Monomers | | |
| Number of | 4 | 4 |
| PPG Monomers | | |
| Fatty Acid | 7 | 7 |
| CH₃(CH₂)ₙCOOH, | | |
| n = ? | | |
| Molecular | 724.8 | 739.8 |
| Weight, MwP | | |
| (g/mol, Dalton) | | |
| nNC | 9 | 9 |
| nOC | 22 | 22 |
| nNO | 0 | 0 |
| nOO | 9 | 9 |
| nNC/(nNC + nOC) | 0.29 | 0.29 |
| nNC/(nNC + nOO) | 0.50 | 0.50 |
| nNO/(nNO + nOC) | 0.00 | 0.00 |
| nNO/(nNO + nOO) | 0.00 | 0.00 |
| Number of | 2 | 2 |
| PEG Monomers | | |
| Number of | 2 | 2 |
| PPG Monomers | | |
| Fatty Acid | 9 | 9 |
| CH₃(CH₂)ₙCOOH, | | |
| n = ? | | |
| Molecular | 504.4 | 519.5 |
| Weight, MwP | | |
| (g/mol, Dalton) | | |
| nNC | 11 | 11 |
| nOC | 10 | 10 |
| nNO | 0 | 0 |
| nOO | 4 | 4 |
| nNC/(nNC + nOC) | 0.52 | 0.62 |
| nNC/(nNC + nOO) | 0.73 | 0.73 |
| nNO/(nNO + nOC) | 0.00 | 0.00 |
| nNO/(nNO + nOO) | 0.00 | 0.00 |
| Number of | 5 | 5 |
| PEG Monomers | | |
| Number of | 2 | 2 |
| PPG Monomers | | |
| Fatty Acid | 11 | 11 |
| CH₃(CH₂)ₙCOOH, | | |
| n = ? | | |
| Molecular | 664.6 | 679.6 |
| Weight, MwP | | |
| (g/mol, Dalton) | | |
| nNC | 13 | 13 |
| nOC | 16 | 16 |
| nNO | 0 | 0 |
| nOO | 7 | 7 |
| nNC/(nNC + nOC) | 0.45 | 0.46 |
| nNC/(nNC + nOO) | 0.65 | 0.65 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 5 | 5 |
| PPG Monomers | | |
| Fatty Acid | 13 | 13 |
| CH₃(CH₂)ₙCOOH, | | |
| n = ? | | |
| Molecular | 646.6 | 661.6 |
| Weight, MwP | | |
| (g/mol, Dalton) | | |
| nNC | 15 | 15 |
| nOC | 15 | 15 |
| nNO | 0 | 0 |
| nOO | 5 | 5 |
| nNC/(nNC + nOC) | 0.50 | 0.50 |
| nNC/(nNC + nOO) | 0.75 | 0.75 |
| nNO/(nNO + nOC) | 0.00 | 0.00 |
| nNO/(nNO + nOO) | 0.00 | 0.00 |
| Number of | 2 | 2 |
| PEG Monomers | | |
| Number of | 2 | 2 |
| PPG Monomers | | |
| Fatty Acid | 15 | 15 |
| CH₃(CH₂)ₙCOOH, | | |
| n = ? | | |
| Molecular | 588.4 | 603.5 |
| Weight, MwP | | |
| (g/mol, Dalton) | | |
| nNC | 17 | 17 |
| nOC | 10 | 10 |
| nNO | 0 | 0 |
| nOO | 4 | 4 |
| nNC/(nNC + nOC) | 0.63 | 0.63 |
| nNC/(nNC + nOO) | 0.81 | 0.81 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 0 | 0 |
| PPG Monomers | | |
| Fatty Acid | 34 | 34 |
| CH₃(CH₂)ₙCOOH | | |
| n = ? | | |
| Molecular | 650.2 | 665.2 |
| Weight, MwP | | |
| (g/mol, Dalton) | | |
| nNC | 36 | 36 |
| nOC | 0 | 0 |
| nNO | 0 | 0 |
| nOO | 0 | 0 |
| nNC/(nNC + nOC) | 1.00 | 1.00 |
| nNC/(nNC + nOO) | 1.00 | 1.00 |
| nNO/(nNO + nOC) | — | — |
| nNO/(nNO + nOO) | — | — |

| | 21st Example | 22nd Example |
|---|---|---|
| IUPAC Name of | Lysine | Glutamine |
| Central Structure | | |
| Structural Formula | 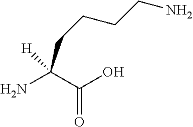 | 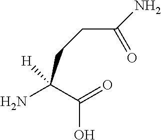 |
| Molecular Weight | 146.19 | 146.15 |
| MwP (g/mol) | | |
| Abbreviation 1 | Lys | Gln |
| Abbreviation 2 | K | Q |
| Residue Property | Basic | Hydrophilic |
| Representative Structure as Side-Chain Structure Being PEG | 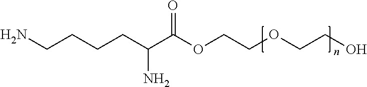 | 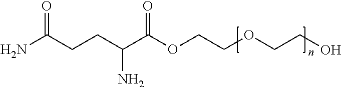 |
| Representative Structure as Side-Chain Structure Being PPG | 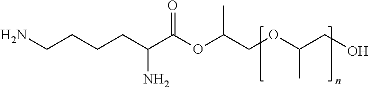 | 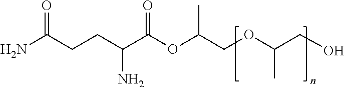 |
| Representative Structure 1 as Side-Chain Structure Being Fatty Acid (CH₃(CH₂)ₙCOOH, n ≥ 3) | 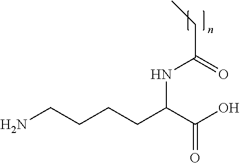 | 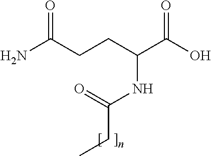 |
| Representative Structure 2 as Side-Chain Structure Being Fatty Acid (CH₃(CH₂)ₙCOOH, | 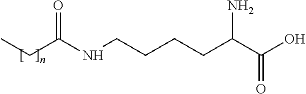 | — |
| n ≥ 3) | | |
| Representative Structure 3 as Side-Chain Structure Being Fatty Acid (CH₃(CH₂)ₙCOOH, n ≥ 3) | 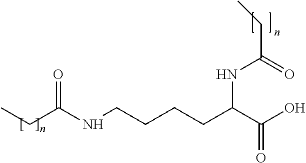 | — |

Side-Chain Structure Being PEG and PPG

| | | |
|---|---|---|
| Number of | 12 | 12 |
| PEG Monomers | | |
| Number of | 0 | 0 |
| PPG Monomers | | |
| Molecular | 674.8 | 674.8 |
| Weight, MwP | | |
| (g/mol, Dalton) | | |
| nNC | 0 | 0 |
| nOC | 24 | 24 |
| nNO | 0 | 0 |
| nOO | 12 | 12 |
| nNC/(nNC + nOC) | 0 | 0 |
| nOC/nOO | 2 | 2 |
| nNO/(nNO + nOC) | 0 | 0 |
| nNO/(nNO + nOO) | 0 | 0 |
| Number of | 0 | 0 |
| PEG Monomers | | |
| Number of | 12 | 12 |
| PPG Monomers | | |
| Molecular | 843.1 | 843.1 |
| Weight, MwP | | |
| (g/mol, Dalton) | | |
| nNC | 0 | 0 |
| nOC | 36 | 36 |
| nNO | 0 | 0 |
| nOO | 12 | 12 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 6 | 6 |
| PEG Monomers | | |
| Number of | 6 | 6 |
| PPG Monomers | | |
| Molecular | 759.0 | 758.9 |
| Weight, MwP | | |
| (g/mol, Dalton) | | |
| nNC | 0 | 0 |
| nOC | 30 | 30 |
| nNO | 0 | 0 |
| nOO | 12 | 12 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 2 | 2 |
| PEG Monomers | | |
| Number of | 5 | 5 |
| PPG Monomers | | |
| Molecular | 524.7 | 524.7 |
| Weight, MwP | | |
| (g/mol, Dalton) | | |
| nNC | 0 | 0 |
| nOC | 19 | 19 |
| nNO | 0 | 0 |
| nOO | 7 | 7 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number Of | 5 | 6 |
| PEG Monomers | | |
| Number of | 2 | 2 |
| PPG Monomers | | |
| Molecular | 482.6 | 482.6 |
| Weight, MwP | | |
| (g/mol, Dalton) | | |
| nNC | 0 | 0 |
| nOC | 16 | 16 |
| nNO | 0 | 0 |
| nOO | 7 | 7 |
| nNC/(nNC + nOC) | 0 | 0 |
| nOC/nOO | 2 | 2 |
| nNO/(nNO + nOC) | 0 | 0 |
| nNO/(nNO + nOO) | 0 | 0 |
| Number of | 5 | 5 |
| PEG Monomers | | |
| Number of | 5 | 5 |
| PPG Monomers | | |
| Molecular | 656.9 | 656.8 |
| Weight, MwP | | |
| (g/mol, Dalton) | | |
| nNC | 0 | 0 |
| nOC | 25 | 25 |
| nNO | 0 | 0 |
| nOO | 10 | 10 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 4 | 4 |
| PEG Monomers | | |
| Number of | 4 | 4 |
| PPG Monomers | | |
| Molecular | 554.7 | 554.7 |
| Weight, MwP | | |
| (g/mol, Dalton) | | |
| nNC | 0 | 0 |
| nOC | 20 | 20 |
| nNO | 0 | 0 |
| nOO | 8 | 8 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 8 | 8 |
| PEG Monomers | | |
| Number of | 8 | 8 |
| PPG Monomers | | |
| Molecular | 963.2 | 963.2 |
| Weight, MwP | | |
| (g/mol, Dalton) | | |
| nNC | 0 | 0 |
| nOC | 40 | 40 |
| nNO | 0 | 0 |
| nOO | 16 | 16 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |

Side-Chain Structure Being PEG, PPG and Fatty Acid

| | | |
|---|---|---|
| Number of | 3 | 3 |
| PEG Monomers | | |
| Number of | 3 | 3 |
| PPG Monomers | | |
| Fatty Acid | 3 | 3 |
| CH₃(CH₂)ₙCOOH, | | |
| n = ? | | |
| Molecular | 536.6 | 536.6 |
| Weight, MwP | | |
| (g/mol, Dalton) | | |
| nNC | 5 | 5 |
| nOC | 15 | 15 |
| nNO | 0 | 0 |
| nOO | 6 | 6 |
| nNC/(nNC + nOC) | 0.25 | 0.25 |
| nNC/(nNC + nOO) | 0.45 | 0.45 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 8 | 8 |
| PPG Monomers | | |
| Fatty Acid | 5 | 5 |
| CH₃(CH₂)ₙCOOH | | |
| n = ? | | |
| Molecular | 722.9 | 722.8 |
| Weight, MwP | | |
| (g/mol, Dalton) | | |
| nNC | 7 | 7 |
| nOC | 24 | 24 |
| nNO | 0 | 0 |
| nOO | 8 | 8 |
| nNC/(nNC + nOC) | 0.23 | 0.23 |
| nNC/(nNC + nOO) | 0.47 | 0.47 |
| nNO/(nNO + nOC) | 0.00 | 0.00 |
| nNO/(nNO + nOO) | 0.00 | 0.00 |
| Number of | 5 | 5 |
| PEG Monomers | | |
| Number of | 4 | 4 |
| PPG Monomers | | |
| Fatty Acid | 7 | 7 |
| CH₃(CH₂)ₙCOOH, | | |
| n = ? | | |
| Molecular | 738.8 | 738.8 |
| Weight, MwP | | |
| (g/mol, Dalton) | | |
| nNO | 9 | 9 |
| nOC | 22 | 22 |
| nNO | 0 | 0 |
| nOO | 9 | 9 |
| nNC/(nNC + nOC) | 0.29 | 0.29 |
| nNC/(nNC + nOO) | 0.50 | 0.50 |
| nNO/(nNO + nOC) | 0.00 | 0.00 |
| nNO/(nNO + nOO) | 0.00 | 0.00 |
| Number of | 2 | 2 |
| PEG Monomers | | |
| Number of | 2 | 2 |
| PPG Monomers | | |
| Fatty Acid | 9 | 9 |
| CH₃(CH₂)ₙCOOH, | | |
| n = ? | | |
| Molecular | 518.5 | 518.5 |
| Weight, MwP | | |
| (g/mol, Dalton) | | |
| nNC | 11 | 11 |
| nOC | 10 | 10 |
| nNO | 0 | 0 |
| nOO | 4 | 4 |
| nNC/(nNC + nOC) | 0.52 | 0.52 |
| nNC/(nNC + nOO) | 0.73 | 0.73 |
| nNO/(nNO + nOC) | 0.00 | 0.00 |
| nNO/(nNO + nOO) | 0.00 | 0.00 |
| Number of | 5 | 5 |
| PEG Monomers | | |
| Number of | 2 | 2 |
| PPG Monomers | | |
| Fatty Acid | 11 | 11 |
| CH₃(CH₂)ₙCOOH, | | |
| n = ? | | |
| Molecular | 678.7 | 678.6 |
| Weight, MwP | | |
| (g/mol, Dalton) | | |
| nNC | 13 | 13 |
| nOC | 16 | 16 |
| nNO | 0 | 0 |
| nOO | 7 | 7 |
| nNC/(nNC + nOC) | 0.46 | 0.45 |
| nNC/(nNC + nOO) | 0.65 | 0.65 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 5 | 5 |
| PPG Monomers | | |
| Fatty Acid | 13 | 13 |
| CH₃(CH₂)ₙCOOH, | | |
| n = ? | | |
| Molecular | 660.6 | 660.6 |
| Weight, MwP | | |
| (g/mol, Dalton) | | |
| nNC | 15 | 15 |
| nOC | 15 | 15 |
| nNO | 0 | 0 |
| nOO | 5 | 5 |
| nNC/(nNC + nOC) | 0.50 | 0.50 |
| nNC/(nNC + nOO) | 0.75 | 0.75 |
| nNO/(nNO + nOC) | 0.00 | 0.00 |
| nNO/(nNO + nOO) | 0.00 | 0.00 |
| Number of | 2 | 2 |
| PEG Monomers | | |
| Number of | 2 | 2 |
| PPG Monomers | | |
| Fatty Acid | 15 | 15 |
| CH₃(CH₂)ₙCOOH, | | |
| n = ? | | |
| Molecular | 602.5 | 602.5 |
| Weight, MwP | | |
| (g/mol, Dalton) | | |
| nNC | 17 | 17 |
| nOC | 10 | 10 |
| nNO | 0 | 0 |
| nOO | 4 | 4 |
| nNC/(nNC + nOC) | 0.63 | 0.63 |
| nNC/(nNC + nOO) | 0.81 | 0.81 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 0 | 0 |
| PPG Monomers | | |
| Fatty Acid | 34 | 34 |
| CH₃(CH₂)ₙCOOH, | | |
| n = ? | | |
| Molecular | 664.3 | 664.2 |
| Weight, MwP | | |
| (g/mol, Dalton) | | |
| nNC | 36 | 36 |
| nOC | 0 | 0 |
| nNO | 0 | 0 |
| nOO | 0 | 0 |
| nNC/(nNC + nOC) | 1.00 | 1.00 |
| nNC/(nNC + nOO) | 1.00 | 1.00 |
| nNO/(nNO + nOC) | — | — |
| nNO/(nNO + nOO) | — | — |

| | 23rd Example | 24th Example |
|---|---|---|
| IUPAC Name of | Arginine | Serine |
| Central Structure | | |
| Structural Formula | 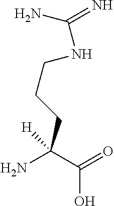 [SMILES: N=C(N)NCCC[C@H](N)C(=O)O] | 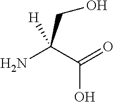 |
| Molecular Weight | 174.2 | 105.09 |
| MwP (g/mol) | | |
| Abbreviation 1 | Arg | Ser |
| Abbreviation 2 | R | S |
| Residue Property | Basic | Hydrophilic |
| Representative Structure as Side-Chain Structure Being PEG | 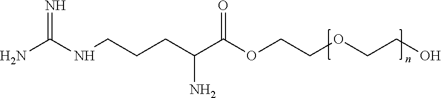 | 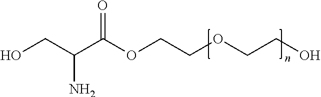 |
| Representative Structure as Side-Chain Structure Being PPG | 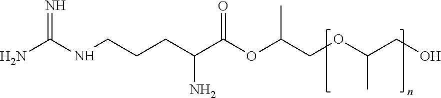 | 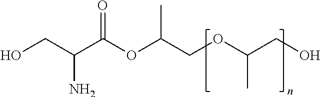 |
| Representative Structure 1 as Side-Chain Structure Being Fatty Acid (CH₃(CH₂)ₙCOOH, n ≥ 3) | 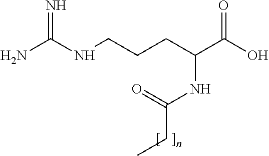 | 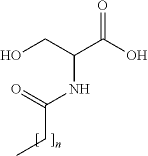 |
| Representative Structure 2 as Side-Chain Structure Being Fatty Acid (CH₃(CH₂)ₙCOOH, | 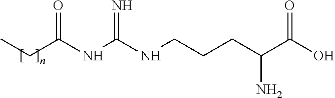 | 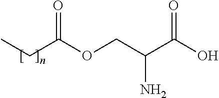 |
| n ≥ 3) | | |
| Representative Structure 3 as Side-Chain Structure Being Fatty Acid (CH₃(CH₂)ₙCOOH, n ≥ 3) | 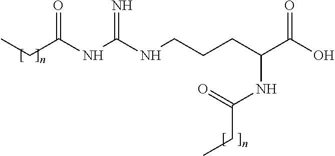 | 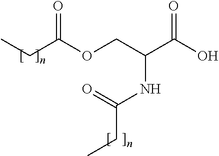 |

Side-Chain Structure Being PEG and PPG

| | | |
|---|---|---|
| Number of | 12 | 12 |
| PEG Monomers | | |
| Number of | 0 | 0 |
| PPG Monomers | | |
| Molecular | 702.8 | 633.7 |
| Weight, MwP | | |
| (g/mol, Dalton) | | |
| nNC | 0 | 0 |
| nOC | 24 | 24 |
| nNO | 0 | 0 |
| nOO | 12 | 12 |
| nNC/(nNC + nOC) | 0 | 0 |
| nOC/nOO | 2 | 2 |
| nNO/(nNO + nOC) | 0 | 0 |
| nNO/(nNO + nOO) | 0 | 0 |
| Number of | 0 | 0 |
| PEG Monomers | | |
| Number of | 12 | 12 |
| PPG Monomers | | |
| Molecular | 871.1 | 802.0 |
| Weight, MwP | | |
| (g/mol, Dalton) | | |
| nNC | 0 | 0 |
| nOC | 36 | 36 |
| nNO | 0 | 0 |
| nOO | 12 | 12 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 6 | 6 |
| PEG Monomers | | |
| Number of | 6 | 6 |
| PPG Monomers | | |
| Molecular | 787.0 | 717.9 |
| Weight, MwP | | |
| (g/mol, Dalton) | | |
| nNC | 0 | 0 |
| nOC | 30 | 30 |
| nNO | 0 | 0 |
| nOO | 12 | 12 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 2 | 2 |
| PEG Monomers | | |
| Number of | 5 | 5 |
| PPG Monomers | | |
| Molecular | 552.7 | 483.6 |
| Weight, MwP | | |
| (g/mol, Dalton) | | |
| nNC | 0 | 0 |
| nOC | 19 | 19 |
| nNO | 0 | 0 |
| nOO | 7 | 7 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 5 | 5 |
| PEG Monomers | | |
| Number of | 2 | 2 |
| PPG Monomers | | |
| Molecular | 510.6 | 441.5 |
| Weight, MwP | | |
| (g/mol, Dalton) | | |
| nNC | 0 | 0 |
| nOC | 16 | 16 |
| nNO | 0 | 0 |
| nOO | 7 | 7 |
| nNC/(nNC + nOC) | 0 | 0 |
| nOC/nOO | 2 | 2 |
| nNO/(nNO + nOC) | 0 | 0 |
| nNO/(nNO + nOO) | 0 | 0 |
| Number of | 5 | 5 |
| PEG Monomers | | |
| Number of | 5 | 5 |
| PPG Monomers | | |
| Molecular | 684.9 | 615.8 |
| Weight, MwP | | |
| (g/mol, Dalton) | | |
| nNC | 0 | 0 |
| nOC | 25 | 25 |
| nNO | 0 | 0 |
| nOO | 10 | 10 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 4 | 4 |
| PEG Monomers | | |
| Number of | 4 | 4 |
| PPG Monomers | | |
| Molecular | 582.7 | 513.6 |
| Weight, MwP | | |
| (g/mol, Dalton) | | |
| nNC | 0 | 0 |
| nOC | 20 | 20 |
| nNO | 0 | 0 |
| nOO | 8 | 8 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 8 | 8 |
| PEG Monomers | | |
| Number of | 8 | 8 |
| PPG Monomers | | |
| Molecular | 991.3 | 922.1 |
| Weight, MwP | | |
| (g/mol, Dalton) | | |
| nNC | 0 | 0 |
| nOC | 40 | 40 |
| nNO | 0 | 0 |
| nOO | 16 | 16 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |

Side-Chain Structure Being PEG, PPG and Fatty Acid

| | | |
|---|---|---|
| Number of | 3 | 3 |
| PEG Monomers | | |
| Number of | 3 | 3 |
| PPG Monomers | | |
| Fatty Acid | 3 | 3 |
| CH₃(CH₂)ₙCOOH, | | |
| n = ? | | |
| Molecular | 564.7 | 495.5 |
| Weight, MwP | | |
| (g/mol, Dalton) | | |
| nNC | 5 | 5 |
| nOC | 15 | 15 |
| nNO | 0 | 0 |
| nOO | 6 | 6 |
| nNC/(nNC + nOC) | 0.25 | 0.25 |
| nNC/(nNC + nOO) | 0.45 | 0.45 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 8 | 8 |
| PPG Monomers | | |
| Fatty Acid | 5 | 5 |
| CH₃(CH₂)ₙCOOH, | | |
| n = ? | | |
| Molecular | 750.9 | 681.8 |
| Weight, MwP | | |
| (g/mol, Dalton) | | |
| nNC | 7 | 7 |
| nOC | 24 | 24 |
| nNO | 0 | 0 |
| nOO | 8 | 8 |
| nNC/(nNC + nOC) | 0.23 | 0.23 |
| nNC/(nNC + nOO) | 0.47 | 0.47 |
| nNO/(nNO + nOC) | 0.00 | 0.00 |
| nNO/(nNO + nOO) | 0.00 | 0.00 |
| Number of | 5 | 5 |
| PEG Monomers | | |
| Number of | 4 | 4 |
| PPG Monomers | | |
| Fatty Acid | 7 | 7 |
| CH₃(CH₂)ₙCOOH, | | |
| n = ? | | |
| Molecular | 766.8 | 697.7 |
| Weight, MwP | | |
| (g/mol, Dalton) | | |
| nNC | 9 | 9 |
| nOC | 22 | 22 |
| nNO | 0 | 0 |
| nOO | 9 | 9 |
| nNC/(nNC + nOC) | 0.29 | 0.29 |
| nNC/(nNC + nOO) | 0.50 | 0.50 |
| nNO/(nNO + nOC) | 0.00 | 0.00 |
| nNO/(nNO + nOO) | 0.00 | 0.00 |
| Number of | 2 | 2 |
| PEG Monomers | | |
| Number of | 2 | 2 |
| PPG Monomers | | |
| Fatty Acid | 9 | 9 |
| CH₃(CH₂)ₙCOOH, | | |
| n = ? | | |
| Molecular | 546.5 | 477.4 |
| Weight, MwP | | |
| (g/mol, Dalton) | | |
| nNC | 11 | 11 |
| nOC | 10 | 10 |
| nNO | 0 | 0 |
| nOO | 4 | 4 |
| nNC/(nNC + nOC) | 0.52 | 0.52 |
| nNC/(nNC + nOO) | 0.73 | 0.73 |
| nNO/(nNO + nOC) | 0.00 | 0.00 |
| nNO/(nNO + nOO) | 0.00 | 0.00 |
| Number of | 5 | 5 |
| PEG Monomers | | |
| Number of | 2 | 2 |
| PPG Monomers | | |
| Fatty Acid | 11 | 11 |
| CH₃(CH₂)ₙCOOH, | | |
| n = ? | | |
| Molecular | 706.7 | 637.6 |
| Weight, MwP | | |
| (g/mol, Dalton) | | |
| nNC | 13 | 13 |
| nOC | 16 | 16 |
| nNO | 0 | 0 |
| nOO | 7 | 7 |
| nNC/(nNC + nOC) | 0.45 | 0.45 |
| nNC/(nNC + nOO) | 0.65 | 0.65 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 5 | 5 |
| PPG Monomers | | |
| Fatty Acid | 13 | 13 |
| CH₃(CH₂)ₙCOOH, | | |
| n = ? | | |
| Molecular | 688.7 | 619.5 |
| Weight, MwP | | |
| (g/mol, Dalton) | | |
| nNC | 15 | 15 |
| nOC | 15 | 15 |
| nNO | 0 | 0 |
| nOO | 5 | 5 |
| nNC/(nNC + nOC) | 0.50 | 0.50 |
| nNC/(nNC + nOO) | 0.75 | 0.75 |
| nNO/(nNO + nOC) | 0.00 | 0.00 |
| nNO/(nNO + nOO) | 0.00 | 0.00 |
| Number of | 2 | 2 |
| PEG Monomers | | |
| Number of | 2 | 2 |
| PPG Monomers | | |
| Fatty Acid | 15 | 15 |
| CH₃(CH₂)ₙCOOH, | | |
| n = ? | | |
| Molecular | 630.5 | 561.4 |
| Weight, MwP | | |
| (g/mol, Dalton) | | |
| nNC | 17 | 17 |
| nOC | 10 | 10 |
| nNO | 0 | 0 |
| nOO | 4 | 4 |
| nNC/(nNC + nOC) | 0.63 | 0.63 |
| nNC/(nNC + nOO) | 0.81 | 0.81 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 0 | 0 |
| PPG Monomers | | |
| Fatty Acid | 34 | 34 |
| CH₃(CH₂)ₙCOOH, | | |
| n = ? | | |
| Molecular | 692.3 | 623.2 |
| Weight, MwP | | |
| (g/mol, Dalton) | | |
| nNC | 36 | 36 |
| nOC | 0 | 0 |
| nNO | 0 | 0 |
| nOO | 0 | 0 |
| nNC/(nNC + nOC) | 1.00 | 1.00 |
| nNC/(nNC + nOO) | 1.00 | 1.00 |
| nNO/(nNO + nOC) | — | — |
| nNO/(nNO + nOO) | — | — |

| | 25th Example | 26th Example |
|---|---|---|
| IUPAC Name of | Threonine | Methionine |
| Central Structure | | |
| Structural Formula | 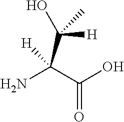 | 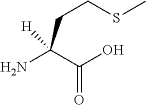 |
| Molecular Weight | 119.12 | 149.21 |
| MwP (g/mol) | | |
| Abbreviation 1 | Thr | Met |
| Abbreviation 2 | T | M |
| Residue Property | Hydrophilic | Hydrophobic |
| Representative Structure as Side-Chain Structure Being PEG | 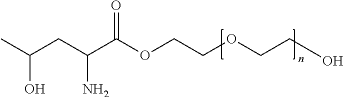 | 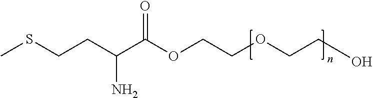 |
| Representative Structure as Side-Chain Structure Being PPG | 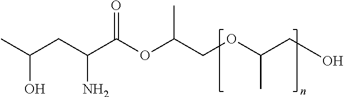 | 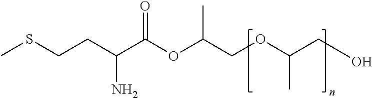 |
| Representative Structure 1 as Side-Chain Structure Being Fatty Acid (CH₃(CH₂)ₙCOOH, n ≥ 3) | 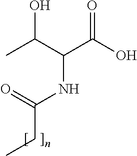 | 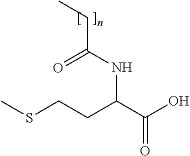 |
| Representative Structure 2 as Side-Chain Structure Being Fatty Acid (CH₃(CH₂)ₙCOOH, n ≥ 3) | 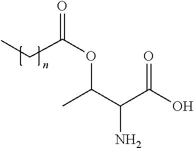 | — |
| Representative Structure 3 as Side-Chain Structure Being Fatty Acid (CH₃(CH₂)ₙCOOH, n ≥ 3) | 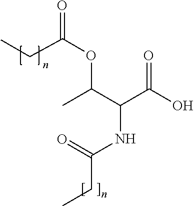 | — |

Side-Chain Structure Being PEG and PPG

| | | |
|---|---|---|
| Number of | 12 | 12 |
| PEG Monomers | | |
| Number of | 0 | 0 |
| PPG Monomers | | |
| Molecular | 647.8 | 677.8 |
| Weight, MwP | | |
| (g/mol, Dalton) | | |
| nNC | 0 | 0 |
| nOC | 24 | 24 |
| nNO | 0 | 0 |
| nOO | 12 | 12 |
| nNC/(nNC + nOC) | 0 | 0 |
| nOC/nOO | 2 | 2 |
| nNO/(nNO + nOC) | 0 | 0 |
| nNO/(nNO + nOO) | 0 | 0 |
| Number of | 0 | 0 |
| PEG Monomers | | |
| Number of | 12 | 12 |
| PPG Monomers | | |
| Molecular | 816.1 | 846.2 |
| Weight, MwP | | |
| (g/mol, Dalton) | | |
| nNC | 0 | 0 |
| nOC | 36 | 36 |
| nNO | 0 | 0 |
| nOO | 12 | 12 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 6 | 6 |
| PEG Monomers | | |
| Number of | 6 | 6 |
| PPG Monomers | | |
| Molecular | 731.9 | 762.0 |
| Weight, MwP | | |
| (g/mol, Dalton) | | |
| nNC | 0 | 0 |
| nOC | 30 | 30 |
| nNO | 0 | 0 |
| nOO | 12 | 12 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 2 | 2 |
| PEG Monomers | | |
| Number of | 5 | 5 |
| PPG Monomers | | |
| Molecular | 497.6 | 527.7 |
| Weight, MwP | | |
| (g/mol, Dalton) | | |
| nNC | 0 | 0 |
| nOC | 19 | 19 |
| nNO | 0 | 0 |
| nOO | 7 | 7 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 5 | 5 |
| PEG Monomers | | |
| Number of | 2 | 2 |
| PPG Monomers | | |
| Molecular | 455.5 | 486.6 |
| Weight, MwP | | |
| (g/mol, Dalton) | | |
| nNC | 0 | 0 |
| nOC | 16 | 16 |
| nNO | 0 | 0 |
| nOO | 7 | 7 |
| nNC/(nNC + nOC) | 0 | 0 |
| nOC/nOO | 2 | 2 |
| nNO/(nNO+ nOC) | 0 | 0 |
| nNO/(nNO + nOO) | 0 | 0 |
| Number of | 5 | 5 |
| PEG Monomers | | |
| Number of | 5 | 5 |
| PPG Monomers | | |
| Molecular | 629.8 | 659.9 |
| Weight, MwP | | |
| (g/mol, Dalton) | | |
| nNC | 0 | 0 |
| nOC | 25 | 25 |
| nNO | 0 | 0 |
| nOO | 10 | 10 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 4 | 4 |
| PEG Monomers | | |
| Number of | 4 | 4 |
| PPG Monomers | | |
| Molecular | 527.6 | 557.7 |
| Weight, MwP | | |
| (g/mol, Dalton) | | |
| nNC | 0 | 0 |
| nOG | 20 | 20 |
| nNO | 0 | 0 |
| nOO | 8 | 8 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 8 | 8 |
| PEG Monomers | | |
| Number of | 8 | 8 |
| PPG Monomers | | |
| Molecular | 936.2 | 966.3 |
| Weight, MwP | | |
| (g/mol, Dalton) | | |
| nNC | 0 | 0 |
| nOC | 40 | 40 |
| nNO | 0 | 0 |
| nOO | 16 | 16 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |

Side-Chain Structure Being PEG, PPG and Fatty Acid

| | | |
|---|---|---|
| Number of | 3 | 3 |
| PEG Monomers | | |
| Number of | 3 | 3 |
| PPG Monomers | | |
| Fatty Acid | | |
| CH₃(CH₂)ₙCOOH, | 3 | 3 |
| n = ? | | |
| Molecular | 509.6 | 539.7 |
| Weight, MwP | | |
| (g/mol, Dalton) | | |
| nNC | 5 | 5 |
| nOC | 15 | 15 |
| nNO | 0 | 0 |
| nOO | 6 | 6 |
| nNC/(nNC + nOC) | 0.25 | 0.25 |
| nNC/(nNC + nOO) | 0.45 | 0.45 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 8 | 8 |
| PPG Monomers | | |
| Fatty Acid | 5 | 5 |
| CH₃(CH₂)ₙCOOH, | | |
| n = ? | | |
| Molecular | 695.8 | 725.9 |
| Weight, MwP | | |
| (g/mol, Dalton) | | |
| nNC | 7 | 7 |
| nOC | 24 | 24 |
| nNO | 0 | 0 |
| nOO | 8 | 8 |
| nNC/(nNC + nOC) | 0.23 | 0.23 |
| nNC/(nNC + nOO) | 0.47 | 0.47 |
| nNO/(nNO + nOC) | 0.00 | 0.00 |
| nNO/(nNO + nOO) | 0.00 | 0.00 |
| Number of | 5 | 5 |
| PEG Monomers | | |
| Number of | 4 | 4 |
| PPG Monomers | | |
| Fatty Acid | 7 | 7 |
| CH₃(CH₂)ₙCOOH, | | |
| n = ? | | |
| Molecular | 711.8 | 741.9 |
| Weight, MwP | | |
| (g/mol, Dalton) | | |
| nNC | 9 | 9 |
| nOC | 22 | 22 |
| nNO | 0 | 0 |
| nOO | 9 | 9 |
| nNC/(nNC + nOC) | 0.29 | 0.29 |
| nNC/(nNC + nOO) | 0.50 | 0.60 |
| nNO/(nNO + nOC) | 0.00 | 0.00 |
| nNO/(nNO + nOO) | 0.00 | 0.00 |
| Number of | 2 | 2 |
| PEG Monomers | | |
| Number of | 2 | 2 |
| PPG Monomers | | |
| Fatty Acid | 9 | 9 |
| CH₃(CH₂)ₙCOOH, | | |
| n = ? | | |
| Molecular | 491.4 | 521.5 |
| Weight, MwP | | |
| (g/mol, Dalton) | | |
| nNC | 11 | 11 |
| nOC | 10 | 10 |
| nNO | 0 | 0 |
| nOO | 4 | 4 |
| nNC/(nNC + nOC) | 0.52 | 0.52 |
| nNC/(nNC + nOO) | 0.73 | 0.73 |
| nNO/(nNO + nOC) | 0.00 | 0.00 |
| nNO/(nNO + nOO) | 0.00 | 0.00 |
| Number of | 5 | 5 |
| PEG Monomers | | |
| Number of | 2 | 2 |
| PPG Monomers | | |
| Fatty Acid | 11 | 11 |
| CH₃(CH₂)ₙCOOH, | | |
| n = ? | | |
| Molecular | 651.6 | 681.7 |
| Weight, MwP | | |
| (g/mol, Dalton) | | |
| nNC | 13 | 13 |
| nOC | 16 | 16 |
| nNO | 0 | 0 |
| nOO | 7 | 7 |
| nNC/(nNC + nOC) | 0.45 | 0.45 |
| nNC/(nNC + nOO) | 0.65 | 0.65 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 5 | 5 |
| PPG Monomers | | |
| Fatty Acid | 13 | 13 |
| CH₃(CH₂)ₙCOOH, | | |
| n = ? | | |
| Molecular | 633.6 | 663.7 |
| Weight, MwP | | |
| (g/mol, Dalton) | | |
| nNC | 15 | 15 |
| nOC | 15 | 15 |
| nNO | 0 | 0 |
| nOO | 5 | 5 |
| nNC/(nNC + nOC) | 0.50 | 0.50 |
| nNC/(nNC + nOO) | 0.75 | 0.75 |
| nNO/(nNO + nOC) | 0.00 | 0.00 |
| nNO/(nNO + nOO) | 0.00 | 0.00 |
| Number of | 2 | 2 |
| PEG Monomers | | |
| Number of | 2 | 2 |
| PPG Monomers | | |
| Fatty Acid | 15 | 15 |
| CH₃(CH₂)ₙCOOH, | | |
| n = ? | | |
| Molecular | 575.4 | 605.5 |
| Weight, MwP | | |
| (g/mol, Dalton) | | |
| nNC | 17 | 17 |
| nOC | 10 | 10 |
| nNO | 0 | 0 |
| nOO | 4 | 4 |
| nNC/(nNC + nOC) | 0.63 | 0.63 |
| nNC/(nNC + nOO) | 0.81 | 0.81 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 0 | 0 |
| PPG Monomers | | |
| Fatty Acid | 34 | 34 |
| CH₃(CH₂)ₙCOOH, | | |
| n = ? | | |
| Molecular | 637.2 | 667.3 |
| Weight, MwP | | |
| (g/mol, Dalton) | | |
| nNC | 36 | 36 |
| nOC | 0 | 0 |
| nNO | 0 | 0 |
| nOO | 0 | 0 |
| nNC/(nNC + nOC) | 1.00 | 1.00 |
| nNC/(nNC + nOO) | 1.00 | 1.00 |
| nNO/(nNO + nOC) | — | — |
| nNO/(nNO + nOO) | — | — |

| | 27th Example | 28th Example |
|---|---|---|
| IUPAC Name of | Cysteine | Proline |
| Central Structure | | |
| Structural Formula | 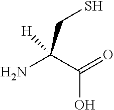 | 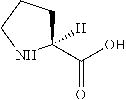 |
| Molecular Weight | 121.16 | 115.13 |
| MwP (g/mol) | | |
| Abbreviation 1 | Cys | Pro |
| Abbreviation 2 | C | P |
| Residue Property | Hydrophilic | Hydrophobic |
| Representative Structure as Side-Chain Structure Being PEG | 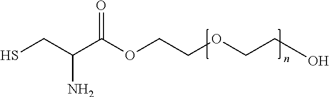 | 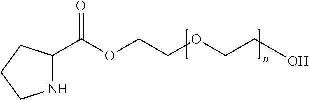 |
| Representative Structure as Side-Chain Structure Being PPG | 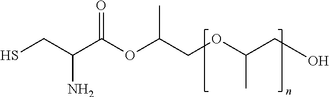 | 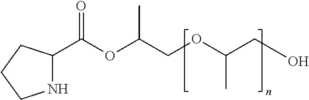 |
| Representative Structure as Side-Chain Structure Being Fatty Acid (CH₃(CH₂)ₙCOOH, n ≥ 3) | 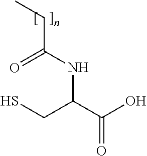 | — |

Side-Chain Structure Being PEG and PPG

| | | |
|---|---|---|
| Number of | 12 | 12 |
| PEG Monomers | | |
| Number of | 0 | 0 |
| PPG Monomers | | |
| Molecular | 649.8 | 643.8 |
| Weight, MwP | | |
| (g/mol, Dalton) | | |
| nNC | 0 | 0 |
| nOC | 24 | 24 |
| nNO | 0 | 0 |
| nOO | 12 | 12 |
| nNC/(nNC + nOC) | 0 | 0 |
| nOC/nOO | 2 | 2 |
| nNO/(nNO + nOC) | 0 | 0 |
| nNO/(nNO + nOO) | 0 | 0 |
| Number of | 0 | 0 |
| PEG Monomers | | |
| Number of | 12 | 12 |
| PPG Monomers | | |
| Molecular | 818.1 | 812.1 |
| Weight, MwP | | |
| (g/mol, Dalton) | | |
| nNC | 0 | 0 |
| nOC | 36 | 36 |
| nNO | 0 | 0 |
| nOO | 12 | 12 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 6 | 6 |
| PEG Monomers | | |
| Number of | 6 | 6 |
| PPG Monomers | | |
| Molecular | 734.0 | 727.9 |
| Weight, MwP | | |
| (g/mol, Dalton) | | |
| nNC | 0 | 0 |
| nOC | 30 | 30 |
| nNO | 0 | 0 |
| nOO | 12 | 12 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 2 | 2 |
| PEG Monomers | | |
| Number of | 5 | 5 |
| PPG Monomers | | |
| Molecular | 499.7 | 493.6 |
| Weight, MwP | | |
| (g/mol, Dalton) | | |
| nNC | 0 | 0 |
| nOC | 19 | 19 |
| nNO | 0 | 0 |
| nOO | 7 | 7 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 5 | 5 |
| PEG Monomers | | |
| Number of | 2 | 2 |
| PPG Monomers | | |
| Molecular | 457.6 | 451.6 |
| Weight, MwP | | |
| (g/mol, Dalton) | | |
| nNC | 0 | 0 |
| nOC | 16 | 16 |
| nNO | 0 | 0 |
| nOO | 7 | 7 |
| nNC/(nNC + nOC) | 0 | 0 |
| nOC/nOO | 2 | 2 |
| nNO/(nNO + nOC) | 0 | 0 |
| nNO/(nNO + nOO) | 0 | 0 |
| Number of | 5 | 5 |
| PEG Monomers | | |
| Number of | 5 | 5 |
| PPG Monomers | | |
| Molecular | 631.8 | 625.8 |
| Weight, MwP | | |
| (g/mol, Dalton) | | |
| nNC | 0 | 0 |
| nOC | 25 | 25 |
| nNO | 0 | 0 |
| nOO | 10 | 10 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 4 | 4 |
| PEG Monomers | | |
| Number of | 4 | 4 |
| PPG Monomers | | |
| Molecular | 529.7 | 523.7 |
| Weight, MwP | | |
| (g/mol, Dalton) | | |
| nNC | 0 | 0 |
| nOC | 20 | 20 |
| nNO | 0 | 0 |
| nOO | 8 | 8 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |
| Number of | 8 | 8 |
| PEG Monomers | | |
| Number of | 8 | 8 |
| PPG Monomers | | |
| Molecular | 938.2 | 932.2 |
| Weight, MwP | | |
| (g/mol, Dalton) | | |
| nNC | 0 | 0 |
| nOC | 40 | 40 |
| nNO | 0 | 0 |
| nOO | 16 | 16 |
| nNC/(nNC + nOC) | 0 | 0 |
| nOC/nOO | 3 | 3 |
| nNO/(nNO + nOC) | 0 | 0 |
| nNO/(nNO + nOO) | 0 | 0 |

Side-Chain Structure Being PEG, PPG and Fatty Acid

| | | |
|---|---|---|
| Number of | 3 | 3 |
| PEG Monomers | | |
| Number of | 3 | 3 |
| PPG Monomers | | |
| Fatty Acid | 3 | 0 |
| CH₃(CH₂)ₙCOOH, | | |
| n = ? | | |
| Molecular | 511.6 | 421.5 |
| Weight, MwP | | |
| (g/mol, Dalton) | | |
| nNC | 5 | 0 |
| nOC | 15 | 15 |
| nNO | 0 | 0 |
| nOO | 6 | 6 |
| nNC/(nNC + nOC) | 0.25 | 0.00 |
| nNC/(nNC + nOO) | 0.45 | 0.00 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 8 | 8 |
| PPG Monomers | | |
| Fatty Acid | 5 | — |
| CH₃(CH₂)ₙCOOH, | | |
| n = ? | | |
| Molecular | 697.9 | 579.8 |
| Weight, MwP | | |
| (g/mol, Dalton) | | |
| nNC | 7 | 0 |
| nOC | 24 | 24 |
| nNO | 0 | 0 |
| nOO | 8 | 8 |
| nNC/(nNC + nOC) | 0.23 | 0.00 |
| nNC/(nNC + nOO) | 0.47 | 0.00 |
| nNO/(nNO + nOC) | 0.00 | 0.00 |
| nNO/(nNO + nOO) | 0.00 | 0.00 |
| Number of | 5 | 5 |
| PEG Monomers | | |
| Number of | 4 | 4 |
| PPG Monomers | | |
| Fatty Acid | 7 | — |
| CH₃(CH₂)ₙCOOH, | | |
| n = ? | | |
| Molecular | 713.8 | 567.7 |
| Weight, MwP | | |
| (g/mol, Dalton) | | |
| nNC | 9 | 0 |
| nOC | 22 | 22 |
| nNO | 0 | 0 |
| nOO | 9 | 9 |
| nNC/(nNC + nOC) | 0.29 | 0.00 |
| nNC/(nNC + nOO) | 0.50 | 0.00 |
| nNO/(nNO + nOC) | 0.00 | 0.00 |
| nNO/(nNO + nOO) | 0.00 | 0.00 |
| Number of | 2 | 2 |
| PEG Monomers | | |
| Number of | 2 | 2 |
| PPG Monomers | | |
| Fatty Acid | 9 | — |
| CH₃(CH₂)ₙCOOH, | | |
| n = ? | | |
| Molecular | 493.5 | 319.4 |
| Weight, MwP | | |
| (g/mol, Dalton) | | |
| nNC | 11 | 0 |
| nOC | 10 | 10 |
| nNO | 0 | 0 |
| nOO | 4 | 4 |
| nNC/(nNC + nOC) | 0.52 | 0.00 |
| nNC/(nNC + nOO) | 0.73 | 0.00 |
| nNO/(nNO + nOC) | 0.00 | 0.00 |
| nNO/(nNO + nOO) | 0.00 | 0.00 |
| Number of | 5 | 5 |
| PEG Monomers | | |
| Number of | 2 | 2 |
| PPG Monomers | | |
| Fatty Acid | 11 | — |
| CH₃(CH₂)ₙCOOH, | | |
| n = ? | | |
| Molecular | 653.6 | 451.6 |
| Weight, MwP | | |
| (g/mol, Dalton) | | |
| nNC | 13 | 0 |
| nOC | 16 | 16 |
| nNO | 0 | 0 |
| nOO | 7 | 7 |
| nNC/(nNC + nOC) | 0.45 | 0.00 |
| nNC/(nNC + nOO) | 0.65 | 0.00 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 5 | 5 |
| PPG Monomers | | |
| Fatty Acid | 13 | — |
| CH₃(CH₂)ₙCOOH, | | |
| n = ? | | |
| Molecular | 635.6 | 405.5 |
| Weight, MwP | | |
| (g/mol, Dalton) | | |
| nNC | 15 | 0 |
| nOC | 15 | 15 |
| nNO | 0 | 0 |
| nOO | 5 | 5 |
| nNC/(nNC + nOC) | 0.50 | 0.00 |
| nNC/(nNC + nOO) | 0.75 | 0.00 |
| nNO/(nNO + nOC) | 0.00 | 0.00 |
| nNO/(nNO + nOO) | 0.00 | 0.00 |
| Number of | 2 | 2 |
| PEG Monomers | | |
| Number of | 2 | 2 |
| PPG Monomers | | |
| Fatty Acid | 15 | — |
| CH₃(CH₂)ₙCOOH, | | |
| n = ? | | |
| Molecular | 577.5 | 319.4 |
| Weight, MwP | | |
| (g/mol, Dalton) | | |
| nNC | 17 | 0 |
| nOC | 10 | 10 |
| nNO | 0 | 0 |
| nOO | 4 | 4 |
| nNC/(nNC + nOC) | 0.63 | 0.00 |
| nNC/(nNC + nOO) | 0.81 | 0.00 |
| nNO/(nNO + nOC) | 0.00 | 0.00 |
| nNO/(nNO + nOO) | 0.00 | 0.00 |
| Number of | 0 | 0 |
| PEG Monomers | | |
| Number of | 0 | 0 |
| PPG Monomers | | |
| Fatty Acid | 34 | 0 |
| CH₃(CH₂)ₙCOOH, | | |
| n = ? | | |
| Molecular | 639.2 | 115.1 |
| Weight, MwP | | |
| (g/mol, Dalton) | | |
| nNC | 36 | 0 |
| nOC | 0 | 0 |
| nNO | 0 | 0 |
| nOO | 0 | 0 |
| nNC/(nNC + nOC) | 1.00 | — |
| nNC/(nNC + nOO) | 1.00 | — |
| nNO/(nNO + nOC) | — | — |
| nNO/(nNO + nOO) | — | — |

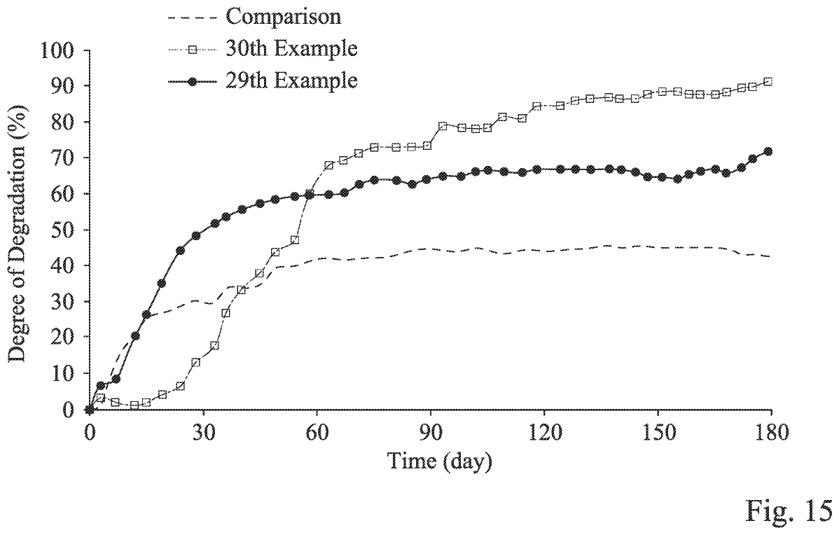

FIG. 15 shows results of biodegradation tests of the comparison, the 29th Example and the 30th Example. The biodegradation tests (ASTM 5338) of the plasticizers with different molecular structures are performed below. The comparison, the 29th Example and the 30th Example are tested, and the degrees of degradation under different days are listed in the following table.

| | Comparison | 29th Example | 30th Example |
|---|---|---|---|
| Central | Citric Acid | Amino Acid | Bezene Derivative |
| Structure | Derivative | Derivative | |
| Product | Acetyl Tributyl | Dioctyl | Ethylene Glycol |
| Name | Citrate | DL-Aspartate | Disalicylate |
| Structural Formula | 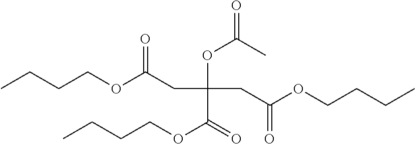 [SMILES: CCCCOC(=O)CC(CC(=O)OCCCC)(OC(C)=O)C(=O)OCCCC] | 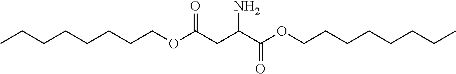 [SMILES: CCCCCCCCOC(=O)CC(N)C(=O)OCCCCCCCC] | 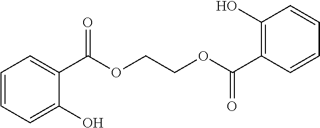 [SMILES: O=C(OCCOC(=O)c1ccccc1O)c1ccccc1O] |
| Reactant 1 | | Aspartate | Salicylic Acid |
| Reactant 2 | | Octanol | Ethylene Glycol |
| days | Degree of Degradation (%) | | |
| 0 | 0 | 0 | 0 |
| 3 | 1.53 | 6.70 | 3.21 |
| 7 | 13.22 | 8.53 | 1.97 |
| 12 | 20.84 | 20.53 | 1.09 |
| 15 | 25.38 | 26.53 | 1.87 |
| 19 | 26.71 | 35.22 | 4.13 |
| 24 | 28.89 | 44.08 | 6.63 |
| 28 | 30.29 | 48.75 | 13.13 |
| 33 | 29.64 | 51.62 | 17.63 |
| 36 | 33.87 | 53.72 | 26.70 |
| 40 | 33.87 | 55.78 | 33.47 |
| 45 | 34.64 | 57.19 | 38.10 |
| 49 | 39.24 | 58.41 | 43.93 |
| 54 | 39.81 | 59.35 | 47.28 |
| 58 | 41.33 | 59.61 | 60.14 |
| 63 | 42.10 | 59.85 | 67.90 |
| 67 | 41.59 | 60.34 | 69.50 |
| 71 | 42.04 | 62.58 | 71.37 |
| 75 | 42.47 | 63.96 | 72.99 |
| 81 | 42.89 | 63.75 | 73.19 |
| 85 | 44.19 | 62.72 | 73.19 |
| 89 | 44.65 | 64.14 | 73.53 |
| 93 | 44.65 | 65.04 | 78.89 |
| 98 | 44.24 | 65.04 | 78.51 |
| 102 | 44.86 | 66.44 | 78.14 |
| 105 | 44.36 | 66.44 | 78.50 |
| 109 | 43.39 | 66.25 | 81.63 |
| 114 | 44.42 | 66.04 | 81.09 |
| 118 | 44.11 | 66.80 | 84.55 |
| 124 | 44.11 | 66.80 | 84.55 |
| 128 | 44.73 | 67.01 | 86.04 |
| 132 | 44.88 | 66.81 | 86.55 |
| 137 | 45.54 | 66.81 | 86.81 |
| 140 | 45.19 | 66.81 | 86.58 |
| 144 | 45.51 | 66.01 | 86.58 |
| 147 | 45.18 | 64.81 | 87.69 |
| 151 | 45.18 | 64.62 | 88.54 |
| 155 | 45.18 | 64.23 | 88.54 |
| 158 | 45.00 | 65.50 | 87.80 |
| 161 | 45.00 | 66.30 | 87.80 |
| 165 | 45.00 | 66.92 | 87.80 |
| 168 | 44.78 | 65.84 | 88.40 |
| 172 | 43.19 | 67.51 | 89.55 |
| 175 | 43.19 | 69.97 | 89.91 |
| 179 | 42.55 | 71.93 | 91.46 |

According to the present disclosure, the biodegradable plasticizer is developed by investigating a novel molecular structure. The plasticizer has the abilities of improving the plastic texture, meeting the requirements for widespread applications, and further enhancing the degradation and cycle of the plastic. The plasticizer includes the first connecting structure and the second connecting structure, includes the benzene derivative and/or the amino acid as the central structure, and is grafted with the side-chain structure of the chain with multiple carbon atoms. The plasticizer can further include the polar functional group to adjust the hydrophilicity and hydrophobicity of the plasticizer. The plasticizer, which reaches desire durability, heat resistance, impact resistance and flexibility, and is harmless to human body, hard to release and low-cost, is developed to meet the target of high biodegradability.

The foregoing description, for purpose of explanation, has been described with reference to specific embodiments and examples. It is to be noted that Tables show different data of the different examples; however, the data of the different examples are obtained from experiments. The embodiments and examples were chosen and described in order to best explain the principles of the disclosure and its practical applications, to thereby enable others skilled in the art to best utilize the disclosure and various embodiments or examples with various modifications as are suited to the particular use contemplated. The embodiments and examples depicted above and the appended drawings are exemplary and are not intended to be exhaustive or to limit the scope of the present disclosure to the precise forms disclosed. Many modifications and variations are possible in view of the above teachings,

What is claimed is:

1. A plasticizer, which is biodegradable, a molecule of the plasticizer comprising:
a central structure comprising at least one of a benzene derivative and at least one amino acid;
at least two connecting structures respectively connected to the central structure, wherein the at least two connecting structures comprise:
a first connecting structure, which is an amine group; and
a second connecting structure, which is a carboxyl group; and
at least one side-chain structure, wherein the at least one side-chain structure is a chain of multiple carbon atoms, the at least one side-chain structure is connected to at least one of the first connecting structure and the second connecting structure, an amide bond is formed as the at least one side-chain structure connected to the amine group, and an ester bond is formed as the at least one side-chain structure connected to the carboxyl group;
wherein a number of carbon atoms of the side-chain structure which is connected to the amine group is nNC, a number of carbon atoms of the side-chain structure which is connected to the carboxyl group is nOC, and the following condition is satisfied:
0≤nNC/(nNC+nOC)≤1.00;
wherein each of nNC and nOC is 0 or positive integer, but nNC and nOC are not 0 at the same time.

2. The plasticizer of claim 1, wherein the central structure is the benzene derivative.

3. The plasticizer of claim 2, wherein the central structure further comprises the at least one amino acid.

4. The plasticizer of claim 1, wherein the central structure is the at least one amino acid.

5. The plasticizer of claim 4, wherein a residue of the at least one amino acid is acidic.

6. The plasticizer of claim 5, wherein the at least one amino acid is aspartate.

7. The plasticizer of claim 4, wherein the central structure comprises at least two of the amino acid.

8. The plasticizer of claim 1, wherein a hydroxyl group is substituted for the amine group of the first connecting structure, and an ether bond is formed as the at least one side-chain structure connected to the hydroxyl group.

9. The plasticizer of claim 1, wherein a molecular weight of the plasticizer is MwP, and the following condition is satisfied:
450 Dalton ≤MwP.

10. The plasticizer of claim 1, further comprising:
at least one polar functional group connected to the at least one side-chain structure, wherein the at least one polar functional group is a carboxyl group, a hydroxyl group, an amine group or a phosphoryl group.

11. The plasticizer of claim 10, wherein the plasticizer comprises at least two of the polar functional group, which are selected from the group consisting of the carboxyl group, the hydroxyl group, the amine group and the phosphoryl group.

12. A plastic product, which is biodegradable, comprising:
the plasticizer of claim 1; and
a polyester plastic, wherein the polyester plastic is a polylactic acid, a polybutylene succinate, a polybutylene adipate-co-terephthalate) or a polyhydroxyalkanoate;
wherein the plasticizer is mixed with the polyester plastic.

13. A plasticizer, which is biodegradable, a molecule of the plasticizer comprising:
at least two central structures, wherein each of the central structures comprises at least one of a benzene derivative and at least one amino acid;
at least two connecting structures, wherein each of the connecting structures is a carboxyl group, and each of the central structures is connected to at least one of the connecting structures; and
a side-chain structure, wherein the side-chain structure is a chain of multiple carbon atoms, each of the central structures is connected to the side-chain structure through the connecting structures, and an ester bond is formed as the side-chain structure connected to each of the carboxyl groups.
